SEQUENCE LISTING (65 sequences)

SEQ ID NO: 1 - RNA, 20 nt, synthetic construct
  source 1..20: mol_type = other RNA
agaccggttctggagacaaa

SEQ ID NO: 2 - RNA, 20 nt, synthetic construct
  source 1..20: mol_type = other RNA
gctctgggactcacattgct

SEQ ID NO: 3 - RNA, 20 nt, synthetic construct
  source 1..20: mol_type = other RNA
tcccagaatcccttgtccct

SEQ ID NO: 4 - RNA, 19 nt, synthetic construct
  source 1..19: mol_type = other RNA
aggcttcctcctcctcacc

SEQ ID NO: 5 - RNA, 20 nt, synthetic construct
  source 1..20: mol_type = other RNA
cgcggtcgccaaaaagaaag

SEQ ID NO: 6 - RNA, 791 nt, Homo sapiens
  source 1..791: mol_type = mRNA
gcgggagggagcgaagcagcgcgggcagcgagcgagatgcagcaccgaggcttcctcctcctcaccctcctcgccctgctggcgctcacctccgcggtcgccaaaaagaaagataaggtgaagaagggcggcccggggagcgagtgcgctgagtgggcctgggggccctgcacccccagcagcaaggattgcggcgtgggtttccgcgagggcacctgcggggcccagacccagcgcatccggtgcagggtgccctgcaactggaagaaggagtttggagccgactgcaagtacaagtttgagaactggggtgcgtgtgatgggggcacaggcaccaaagtccgccaaggcaccctgaagaaggcgcgctacaatgctcagtgccaggagaccatccgcgtcaccaagccctgcacccccaagaccaaagcaaaggccaaagccaagaaagggaagggaaaggactagacgccaagcctggatgccaaggagcccctggtgtcacatggggcctggcccacgccctccctctcccaggcccgagatgtgacccaccagtgccttctgtctgctcgttagctttaatcaatcatgccctgccttgtccctctcactccccagccccacccctaagtgcccaaagtggggagggacaagggattctgggaagcttgagcctcccccaaagcaatgtgagtcccagagcccgcttttgttcttccccacaattccattactaagaaacacatcaaataaactgactttttccccccaaaaaaaaaaaaaaaaaa

SEQ ID NO: 7 - DNA, 56 bp, Homo sapiens
  source 1..56: mol_type = genomic DNA
gcagggtgccctgcaactggaagaaggagtttggaggtgaggcggggcgcagtcag

SEQ ID NO: 8 - DNA, 35 bp, Homo sapiens
  source 1..35: mol_type = genomic DNA
gcagggtgccctgcaactggaagaaggagtttgga

SEQ ID NO: 9 - DNA, 30 bp, Homo sapiens
  source 1..30: mol_type = genomic DNA
gcagggtgccctgcaactggaagaaggagt

SEQ ID NO: 10 - DNA, 22 bp, Homo sapiens
  source 1..22: mol_type = genomic DNA
caactggaagaaggagtttgga

SEQ ID NO: 11 - DNA, 33 bp, Homo sapiens
  source 1..33: mol_type = genomic DNA
caactggaagaaggagtttggaggtgaggcggg

SEQ ID NO: 12 - DNA, 41 bp, Homo sapiens
  source 1..41: mol_type = genomic DNA
actggaagaaggagtttggaggtgaggcggggcgcagtcag

SEQ ID NO: 13 - DNA, 31 bp, Homo sapiens
  source 1..31: mol_type = genomic DNA
actggaagaaggagtttggaggtgaggcggg

SEQ ID NO: 14 - DNA, 30 bp, Homo sapiens
  source 1..30: mol_type = genomic DNA
gagtttggaggtgaggcggggcgcagtcag

SEQ ID NO: 15 - DNA, 114 bp, Homo sapiens
  source 1..114: mol_type = genomic DNA
ggcaccctgaagaaggcgcgctacaatgctcagtgccaggagaccatccgcgtcaccaagccctgcacccccaagaccaaagcaaaggccaaaggtcagcgaaaggagaagggg

SEQ ID NO: 16 - DNA, 67 bp, Homo sapiens
  source 1..67: mol_type = genomic DNA
ggcaccctgaagaaggcgcgctacaatgctcagtgccaggagaccatccgcgtcaccaagccctgca

SEQ ID NO: 17 - DNA, 35 bp, Homo sapiens
  source 1..35: mol_type = genomic DNA
ggcaccctgaagaaggcgcgctacaatgctcagtg

SEQ ID NO: 18 - DNA, 35 bp, Homo sapiens
  source 1..35: mol_type = genomic DNA
gtgccaggagaccatccgcgtcaccaagccctgca

SEQ ID NO: 19 - DNA, 35 bp, Homo sapiens
  source 1..35: mol_type = genomic DNA
aagcaaaggccaaaggtcagcgaaaggagaagggg

SEQ ID NO: 20 - DNA, 25 bp, Homo sapiens
  source 1..25: mol_type = genomic DNA
cgcgcgttgtagataaggtgaagaa

SEQ ID NO: 21 - DNA, 25 bp, Homo sapiens
  source 1..25: mol_type = genomic DNA
gtagataaggtgaagaagggcggcc

SEQ ID NO: 22 - DNA, 25 bp, Homo sapiens
  source 1..25: mol_type = genomic DNA
tttccgcgagggcacctgcggggcc

SEQ ID NO: 23 - DNA, 25 bp, Homo sapiens
  source 1..25: mol_type = genomic DNA
ctgcaactggaagaaggagtttgga

SEQ ID NO: 24 - DNA, 20 bp, Homo sapiens
  source 1..20: mol_type = genomic DNA
caactggaagaaggagtttg

SEQ ID NO: 25 - DNA, 25 bp, Homo sapiens
  source 1..25: mol_type = genomic DNA
agaaggagtttggaggtgaggcggg

SEQ ID NO: 26 - DNA, 25 bp, Homo sapiens
  source 1..25: mol_type = genomic DNA
ctctctcgccagccgactgcaagta

SEQ ID NO: 27 - DNA, 25 bp, Homo sapiens
  source 1..25: mol_type = genomic DNA
ccagccgactgcaagtacaagtttg

SEQ ID NO: 28 - DNA, 25 bp, Homo sapiens
  source 1..25: mol_type = genomic DNA
cctgaagaaggcgcgctacaatgct

SEQ ID NO: 29 - DNA, 25 bp, Homo sapiens
  source 1..25: mol_type = genomic DNA
aggagaccatccgcgtcaccaagcc

SEQ ID NO: 30 - DNA, 25 bp, Homo sapiens
  source 1..25: mol_type = genomic DNA
aaggccaaaggtcagcgaaaggaga

SEQ ID NO: 31 - DNA, 25 bp, Homo sapiens
  source 1..25: mol_type = genomic DNA
gcagggtgccctgcaactggaagaa

SEQ ID NO: 32 - DNA, 25 bp, Homo sapiens
  source 1..25: mol_type = genomic DNA
gtgccctgcaactggaagaaggagt

SEQ ID NO: 33 - DNA, 25 bp, Homo sapiens
  source 1..25: mol_type = genomic DNA
actggaagaaggagtttggaggtga

SEQ ID NO: 34 - DNA, 25 bp, Homo sapiens
  source 1..25: mol_type = genomic DNA
gagtttggaggtgaggcggggcgca

SEQ ID NO: 35 - DNA, 25 bp, Homo sapiens
  source 1..25: mol_type = genomic DNA
gagtttggaggtgaggcggggcgca

SEQ ID NO: 36 - DNA, 25 bp, Homo sapiens
  source 1..25: mol_type = genomic DNA
ggcaccctgaagaaggcgcgctaca

SEQ ID NO: 37 - DNA, 25 bp, Homo sapiens
  source 1..25: mol_type = genomic DNA
agaaggcgcgctacaatgctcagtg

SEQ ID NO: 38 - DNA, 25 bp, Homo sapiens
  source 1..25: mol_type = genomic DNA
gtgccaggagaccatccgcgtcacc

SEQ ID NO: 39 - DNA, 25 bp, Homo sapiens
  source 1..25: mol_type = genomic DNA
accatccgcgtcaccaagccctgca

SEQ ID NO: 40 - DNA, 25 bp, Homo sapiens
  source 1..25: mol_type = genomic DNA
aagcaaaggccaaaggtcagcgaaa

SEQ ID NO: 41 - DNA, 25 bp, Homo sapiens
  source 1..25: mol_type = genomic DNA
caaaggtcagcgaaaggagaagggg

SEQ ID NO: 42 - DNA, 25 bp, Homo sapiens
  source 1..25: mol_type = genomic DNA
agtgccaggagaccatccgcgtcac

SEQ ID NO: 43 - RNA, 25 nt, synthetic construct
  source 1..25: mol_type = other RNA
ttcttcaccttatctacaacgcgcg

SEQ ID NO: 44 - RNA, 25 nt, synthetic construct
  source 1..25: mol_type = other RNA
ggccgcccttcttcaccttatctac

SEQ ID NO: 45 - RNA, 25 nt, synthetic construct
  source 1..25: mol_type = other RNA
ggccccgcaggtgccctcgcggaaa

SEQ ID NO: 46 - RNA, 25 nt, synthetic construct
  source 1..25: mol_type = other RNA
tccaaactccttcttccagttgcag

SEQ ID NO: 47 - RNA, 20 nt, synthetic construct
  source 1..20: mol_type = other RNA
aaactccttcttccagttgc

SEQ ID NO: 48 - RNA, 25 nt, synthetic construct
  source 1..25: mol_type = other RNA
cccgcctcacctccaaactccttct

SEQ ID NO: 49 - RNA, 25 nt, synthetic construct
  source 1..25: mol_type = other RNA
tacttgcagtcggctggcgagagag

SEQ ID NO: 50 - RNA, 25 nt, synthetic construct
  source 1..25: mol_type = other RNA
caaacttgtacttgcagtcggctgg

SEQ ID NO: 51 - RNA, 25 nt, synthetic construct
  source 1..25: mol_type = other RNA
agcattgtagcgcgccttcttcagg

SEQ ID NO: 52 - RNA, 25 nt, synthetic construct
  source 1..25: mol_type = other RNA
ggcttggtgacgcggatggtctcct

SEQ ID NO: 53 - RNA, 25 nt, synthetic construct
  source 1..25: mol_type = other RNA
tctcctttcgctgacctttggcctt

SEQ ID NO: 54 - RNA, 25 nt, synthetic construct
  source 1..25: mol_type = other RNA
ttcttccagttgcagggcaccctgc

SEQ ID NO: 55 - RNA, 25 nt, synthetic construct
  source 1..25: mol_type = other RNA
actccttcttccagttgcagggcac

SEQ ID NO: 56 - RNA, 25 nt, synthetic construct
  source 1..25: mol_type = other RNA
tcacctccaaactccttcttccagt

SEQ ID NO: 57 - RNA, 25 nt, synthetic construct
  source 1..25: mol_type = other RNA
tgcgccccgcctcacctccaaactc

SEQ ID NO: 58 - RNA, 25 nt, synthetic construct
  source 1..25: mol_type = other RNA
ctgactgcgccccgcctcacctcca

SEQ ID NO: 59 - RNA, 25 nt, synthetic construct
  source 1..25: mol_type = other RNA
tgtagcgcgccttcttcagggtgcc

SEQ ID NO: 60 - RNA, 25 nt, synthetic construct
  source 1..25: mol_type = other RNA
cactgagcattgtagcgcgccttct

SEQ ID NO: 61 - RNA, 25 nt, synthetic construct
  source 1..25: mol_type = other RNA
ggtgacgcggatggtctcctggcac

SEQ ID NO: 62 - RNA, 25 nt, synthetic construct
  source 1..25: mol_type = other RNA
tgcagggcttggtgacgcggatggt

SEQ ID NO: 63 - RNA, 25 nt, synthetic construct
  source 1..25: mol_type = other RNA
tttcgctgacctttggcctttgctt

SEQ ID NO: 64 - RNA, 25 nt, synthetic construct
  source 1..25: mol_type = other RNA
ccccttctcctttcgctgacctttg

SEQ ID NO: 65 - RNA, 25 nt, synthetic construct
  source 1..25: mol_type = other RNA
gtgacgcggatggtctcctggcact

(19) United States
(12) Patent Application Publication
(10) Pub. No.: US 2025/0297257 A1
(43) Pub. Date: Sep. 25, 2025

(54) SPLICE-SWITCHING OLIGONUCLEOTIDES

(71) Applicant: Lyramid Limited, Sydney, New South Wales (AU)

(72) Inventors: Graham ROBERTSON, Northmead, New South Wales (AU); Jessica CALE, Coolbellup, Western Australia (AU); Steve WILTON, Applecross, Western Australia (AU); May AUNG-HTUT, Willeton, Western Australia (AU)

(21) Appl. No.: 18/847,933

(22) PCT Filed: Mar. 20, 2023
(86) PCT No.: PCT/AU2023/050195

§ 371 Date: Sep. 17, 2024

(30) Foreign Application Priority Data
Mar. 18, 2022 (AU) 2022900678

(51) Int. Cl. C12N 15/113

(52) U.S. Cl. CPC C12N 15/1136; C12N 2310/11; C12N 2310/315; C12N 2310/321; C12N 2310/3231; C12N 2310/3233; C12N 2320/33

(57) ABSTRACT

The present disclosure relates generally to novel splice-switching oligonucleotides (SSOs) capable of inducing exon skipping in human midkine, compositions comprising same, and use thereof to treat individuals with a midkine-related disease or disorder.

### RELATED APPLICATION DATA

The present application is a U.S. National Phase Application under 35 U.S.C. § 371 of International Application No. PCT/AU2023/050195, filed Mar. 20, 2023, which claims benefit of Australian Patent Application No. 2022900678 entitled ‘Splice-switching oligonucleotides’ filed Mar. 18, 2022. The entire contents of which is hereby incorporated by reference.

### SEQUENCE LISTING

The present application is filed with a Sequence Listing in electronic form. The entire contents of the Sequence Listing are hereby incorporated by reference.

### TECHNICAL FIELD

The present disclosure relates to novel splice-switching oligonucleotides (SSOs) capable of inducing exon skipping in human midkine, compositions comprising same, and use thereof to treat individuals with a midkine-related disease or disorder.

### BACKGROUND

Midkine (MDK) is a heparin-binding growth factor found as a product of a gene transiently expressed in the stage of retinoic acid-induced differentiation of embryonal carcinoma (EC) cells and is a polypeptide of 13 kDa in molecular weight rich in basic amino acids and cysteine (Kadomatsu. et al. (1988) Biochem. Biophys. Res. Commun., 151:1312-1318; Tomokura et al. (1999) J. Biol. Chem, 265:10765-10770; Muramatsu T (2014) Brit J Pharmacol 171:814-826).

MDK is known to have various biological activities. For example, it is known that MDK expression is increased in human cancer cells. This increase in expression has been confirmed in various cancers such as esophageal cancer, thyroid cancer, urinary bladder cancer, colon cancer, stomach cancer, pancreatic cancer, thoracic cancer, liver cancer, lung cancer, breast cancer, neuroblastoma, glioblastoma, mesothelioma, renal cancer, head and neck cancer, melanoma, uterine/cervical cancer, ovarian cancer, osteosarcoma, chronic lymphocytic leukaemia and Wilms tumour [Muramatsu (2002) J. Biochem. 132:359-371; Jones (2014) Brit J Pharm 171:2925-2939]. Moreover, MDK enhances the survival and migration of cancer cells, promotes angiogenesis, as well as contributing to cancer progression and metastasis. MDK is also a major determinant of response to cancer treatment, including chemotherapy and immunotherapy.

MDK is also known to play a central role in regulating immune and inflammatory responses [Heradon G et al (2019) Frontier Pharmacol 10:377; Aynacioglu A S et al (2018) Modern Rheumatology 29:567-571; Sorrelle N et al (2017) J Leukoc Biol 102:277-286]. For example, it is known that neointimal formation after vascular injury and nephritis onset during ischemic injury are suppressed in knockout mice deficient in MDK genes. Moreover, it is also known that rheumatism models and postoperative adhesions are significantly suppressed in such knockout mice (WO2000/10608; WO2004/078210). Thus, MDK is known to participate in inflammatory diseases and autoimmune disorders such as arthritis (both Rheumatoid and Osteoarthritis), postoperative adhesion, inflammatory bowel disease, autoimmune myocarditis, chronic kidney disease, psoriasis, lupus, asthma, and multiple sclerosis involving T regulatory cell dysfunction [Takeuchi H (2014) Brit J Pharmacol 171:931-935]. Furthermore, MDK is known to promote the migration, activation and functional orientation of inflammatory cells such as macrophages or neutrophils. Since recruitment and deleterious behaviour of neutrophils and macrophages are necessary for the establishment of inflammatory responses in diseased tissues, deficiency or blockade of MDK action prevents diseases based on inflammation in animal models (WO1999/03493). However, there are no midkine-based therapies that have progressed beyond preclinical experimental testing. This limitation is exemplified by monoclonal antibodies targeting midkine that have only shown benefit when administered in prophylactic mode and failed in treatment mode once the disease or tumour is established.

Accordingly, there remains a need for new compositions and methods with improved ability to modulate, inhibit or reduce the abundance and activity of functional MDK.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

The present disclosure is based, inter alia, on a recognition by the inventors that there is a need for novel therapeutic strategies to target MDK activity or function and treat conditions associated with MDK action. To this end, the inventors have developed novel antisense splice-switching oligonucleotides (SSOs) capable of inducing exon skipping of human midkine pre-mRNA hence blocking synthesis of the mature mRNA encoding the native midkine protein present in diseased tissues. The resulting MDK mRNAs are translated into forms of MDK protein that lack critical functional domains, thereby reducing the abundance of the full length, biologically active MDK protein. In addition, the inventors have undertaken microwalking of lead candidate SSOs and found that, for both exon 3 and exon 4, the SSOs targeting regions closest to the donor splice site were the most effective at inducing exon skipping. The inventors have also demonstrated that the efficacy of SSOs against MDK may be improved when used as a cocktail. Collectively, these findings by the inventors provide novel agents and strategies for treating, preventing or inhibiting midkine-related diseases or disorders.

Thus, the present disclosure provides a splice-switching oligonucleotide (SSO) which is between 10 and 50 nucleotides in length, the SSO comprising a nucleotide sequence of at least 10 contiguous nucleotides which is substantially complementary to a target region of corresponding length within a pre-mRNA sequence of human midkine. In some examples, the nucleotide sequence will be less than 26 nucleotides in length. For example, a suitable sequence may be in the range of 20-25 nucleotides in length. For example, the sequence may be 25 nucleotides in length.

In one example, the SSO comprises a nucleotide sequence comprising 20-25 contiguous nucleotides which is substantially complementary to the target region.

In one example, the SSO specifically hybridises to the target region of corresponding length within the pre-mRNA sequence of human midkine to disrupt splicing and thereby induce exon skipping of human midkine. For example, the SSO may disrupt splicing and thereby induce exon skipping of exon 1, 2, 3, 4 or 5. For example, the SSO specifically hybridises to the target region of corresponding length within the pre-mRNA sequence of human midkine to disrupt splicing and thereby induce exon skipping of exon 3 or exon 4 of human midkine.

In one example, the SSO specifically hybridises to the target region of corresponding length within the pre-mRNA sequence of human midkine to disrupt splicing and thereby induce exon skipping of exon 3. In accordance with this example, exon skipping results in a midkine mRNA transcript lacking exon 3.

In another example, the SSO specifically hybridises to the target region of corresponding length within the pre-mRNA sequence of human midkine to disrupt splicing and thereby induce exon skipping of exon 4. In accordance with this example, exon skipping results in a midkine mRNA transcript lacking exon 4.

In one example, the target region within a pre-mRNA sequence of human midkine comprises a sequence selected from the group consisting of SEQ ID NO: 7-42. In accordance with this example, the SSO comprises a nucleotide sequence which is substantially complementary to a target region of corresponding length within a pre-mRNA sequence set forth in SEQ ID NO: 7-42. For example, the SSO may comprise a nucleotide sequence which is complementary to a target region set forth in any one of SEQ ID NOs: 7-42 with the exception of 4 base pair mismatches. For example, the SSO may comprise a nucleotide sequence which is complementary to a target region set forth in any one of SEQ ID NOs: 7-42 with the exception of 3 base pair mismatches. For example, the SSO may comprise a nucleotide sequence which is complementary to a target region set forth in any one of SEQ ID NOs: 7-42 with the exception of 2 base pair mismatches. For example, the SSO may comprise a nucleotide sequence which is complementary to a target region set forth in any one of SEQ ID NOs: 7-42 with the exception of 1 base pair mismatch. In particular example, the SSO comprises a nucleotide sequence which is 100% complementary to a target region of equivalent length within a sequence set forth in any one of SEQ ID NOs: 7-42.

According to examples in which the SSO induces skipping of exon 3, the target region may be selected from the group of sequences set forth in SEQ ID NO: 7-14, 20-25 and 31-35. For example, the target region may be selected from the group of sequences set forth in SEQ ID NOs: 7-14. For example, the target region may be selected from the group of sequences set forth in SEQ ID NO: 20-25 and 31-35. In one particular example, the target region is set forth in SEQ ID NO: 23.

According to examples in which the SSO induces skipping of exon 4, the target region may be selected from the group of sequences set forth in SEQ ID NO: 15-19, 26-30 and 36-42. For example, the target region may be selected from the group of sequences set forth in SEQ ID NO: 15-19. For example, the target region may be selected from the group of sequence set forth in SEQ ID NO: 26-30 and 36-42. In one particular example, the target region is selected from the sequence set forth in SEQ ID NO: 37, 38 and 42.

In one example, the SSO may comprise a nucleotide sequence selected from the group of sequences set forth in SEQ ID NO: 43-65. According to examples in which the SSO induces skipping of exon 3, the SSO comprises a nucleotide sequence selected from the group of sequences set forth in SEQ ID NO: 43-48 and 54-58. For example, the SSO may comprise a nucleotide sequence set forth in SEQ ID NO: 46. According to examples in which the SSO induces skipping of exon 4, the SSO comprises a nucleotide sequence selected from the group of sequences set forth in SEQ ID NO: 49-53 and 59-65. For example, the SSO may comprise a nucleotide sequence selected from the group of sequences set forth in SEQ ID NO: 60, 61 and 65.

In each of the foregoing examples, one or more of the nucleotides within the SSO may be modified. In some examples, all of the nucleotides within the SSO are modified. Examples of modified nucleotides useful in the SSOs of the disclosure include, but are not limited to, those which comprise a 2′-O-methyl, 2′-O-methoxyethoxy, 2′-fluoro, 2′-allyl, 2′-O-[2-(methylamino)-2-oxoethyl], 4′-thio, 4′-CH2-O-2′-bridge, 4′-(CH2)2-O-2′-bridge, 2′-LNA, 2′-amino, fluoroarabinonucleotide, threose nucleic acid or 2′-O—(N-methlycarbamate).

The modified SSO may be a 2′-0-methyl phosphorothioate (2′-OMe-PS) SSO, a 2′-0-methoxyethyl (2′MOE) SSO, a locked nucleic acid (LNA) SSO, a morpholino oligonucleotide SSO, a phosphorodiamidate morpholino (PMO) SSO or an SSO comprising any combination of the aforementioned nucleotide chemistries. In example, the modified SSO comprises a 2′O-methyl and a phosphorothioate backbone. In another example, the modified SSO comprises a phosphorodiamidate morpholino backbone.

The present disclosure also provides a pharmaceutical composition comprising one or more SSOs as described herein. In some examples, the composition further comprises one or more pharmaceutically acceptable carriers or diluents.

In some examples, the pharmaceutical composition comprises a plurality of SSOs as described herein (e.g., 2 or more of the SSO described herein). For example, the pharmaceutical composition may comprise a plurality (e.g., 2, or 3, or 4 or more) of SSOs as described herein, each comprising a nucleotide sequence which is substantially complementary to a target region of corresponding length within a pre-mRNA sequence set forth in one of SEQ ID NOs: 7-42. For example, the pharmaceutical composition may comprise two or more of the SSOs comprising nucleotide sequences selected from the group of sequences set forth in SEQ ID NOs: 43-65.

In some examples, the plurality of SSOs within the pharmaceutical composition comprises at least one SSO which induces skipping of exon 3 and at least one SSO which induces skipping of exon 4. For example, the plurality of SSOs may comprise at least one SSO comprising a sequence selected from the group of sequences set forth in SEQ ID NOs: 43-48 and 54-58 and at least one SSO comprising a sequence selected from the group of sequences set forth in SEQ ID NOs: 49-53 and 59-65.

The present disclosure also provides a method for inhibiting an interaction between human midkine and a ligand thereof on the surface of or in a cell, said method comprising exposing the cell to one or more SSOs described herein or to the pharmaceutical composition described herein.

The present disclosure also provides a method for inhibiting human midkine activity in a cell, said method comprising exposing the cell to one or more SSOs described herein or to the pharmaceutical composition described herein.

The present disclosure also provides a method for treating or preventing a midkine-related disease or disorder in a subject in need thereof, said method comprising administering to the subject one or more SSOs described herein or the pharmaceutical composition described herein.

The present disclosure also provides for use of one or more SSOs described herein, or the pharmaceutical composition described herein, in the preparation of a medicament for treatment or prevention of a midkine-related disease or disorder selected from an autoimmune disease, cancer, or an inflammatory disease in a subject in need thereof. In some examples, the medicament may further comprise a chemotherapeutic agent.

The present disclosure also provides for use of one or more SSOs described herein, or the pharmaceutical composition described herein, to treat or prevent a midkine-related disease or disorder selected from an autoimmune disease, cancer, or an inflammatory disease in a subject in need thereof.

In one example, the subject to which the SSO, plurality of SSOs and/or pharmaceutical composition of the disclosure is/are administered has already received treatment with another therapeutic agent for treating a midkine-related disease or disorder. For example, the subject and/or the midkine-related disease or disorder to be treated may be refractory or resistant to treatment with the other agent known for treating a midkine-related disease or disorder. In one example, the other agent known for treating a midkine-related disease or disorder is a chemotherapeutic agent.

In another example, the SSO, plurality of SSOs and/or pharmaceutical composition of the disclosure is administered in combination with another therapeutic agent known for treating a midkine-related disease or disorder i.e., as an adjunctive therapy.

Treatment of a midkine-related disease or disorder in accordance with any example described herein, may comprise one or more of inhibiting, reducing or preventing midkine activity in the subject and/or reducing severity of symptoms associated with a midkine-related disease or disorder. In one example, the medicament will reduce midkine gene transcription products in the subject to which the medicament is administered.

Examples of midkine-related diseases or disorders that can be inhibited, treated or prevented include, but are not limited to, autoimmune diseases, cancer, or inflammatory diseases. In one example, the midkine-related disease or disorder is cancer. In another example, the midkine-related disease or disorder is an inflammatory disease.

### BRIEF DESCRIPTION OF THE DRAWINGS

FIG. 1 a) shows the SSO nomenclature. SSO name includes information on the gene, species, exon number, and sequence coordinates relative to splice donor and acceptor sites; and b) shows the PCR primer nomenclature. PCR primer ID includes species, gene, target, primer direction and additional information.

FIG. 2 is the midkine (MDK) transcripts reported on Ensembl. Each box represents an exon, and the solid black line represents introns. Chevron sides indicate exons bounded by partial codons. Primers are shown in purple.

FIG. 3 is the Midkine genomic sequence (NC_000011.10) annotated with exon regions numbered based on MANE select transcript T203/transcript variant 3. Primers are shown in purple.

FIG. 4 is the optimisation of six PCR primer sets by alteration of PCR methods, annealing temperature, cycle number, and cell type.

FIG. 5 provides the locations of exon splice enhancer (ESE) and exon splice silencer (ESS) motifs within MDK mRNA predicted by Spliceaid and SSO annealing sites targeted to remove a) exon 3 and b) exon 4. Relative predicted splice factor binding site motif scores are indicated on the y-axis with positive values above the mRNA sequence indicative of splice enhancer motifs while negative values indicate splice silencer motifs. Exonic sequences are shown in upper case letters, and intronic sequences are depicted in lower case. The first generation 2′OMe-PS SSOs are shown in black, the second generation microwalked SSOs in blue and the single 20-mer SSO in purple.

FIG. 6 provides evaluation of SSOs in Huh7 cells. RT-PCR analysis of MDK transcripts after Lipofectamine 3000 transfection with SSOs targeting MDK mRNA a) exon 3 and b) exon 4. Transfection concentrations (50-200 nM) are indicated above the gel image. Relative abundance (%) of amplicons are shown in the graph beside each gel image. GTC, Gene Tools control; Neg, no template PCR control; UT, untreated; bp, base pairs; FL, full-length amplicon; nM, nanomolar; i, intron.

FIG. 7 provides evaluation of two-SSO cocktails in Huh7 cells. RT-PCR analysis of MDK transcripts after Lipofectamine 3000 transfection with two-SSO cocktails targeting MDK mRNA a) exon 3 and b) exon 4. Transfection concentrations are indicated above the gel image. Relative abundance (%) of amplicons are shown in the graph below each gel image. GTC, Gene Tools control; Neg, no template PCR control; UT, untreated; bp, base pairs; FL, full-length amplicon; nM, nanomolar; i, intron.

FIG. 8 provides evaluation of lower SSO concentrations in Huh7 cells. RT-PCR analysis of MDK transcripts after Lipofectamine 3000 transfection with SSOs targeting MDK a) exon 3 and b) exon 4. Transfection concentrations are indicated above the gel image. Relative abundance (%) of amplicons lacking exon 3 (Δ3) or exon 4 (Δ4) are shown below each gel image. GTC, Gene Tools control; Neg, no template PCR control; UT, untreated; bp, base pairs; FL, full-length amplicon; nM, nanomolar.

FIG. 9 provides evaluation of SSOs microwalked around original sequences in Huh7 cells. RT-PCR analysis of MDK transcripts after Lipofectamine 3000 transfection with SSOs targeting MDK a) exon 3 and b) exon 4. The original sequence is shown in black, the microwalked sequences in blue and the 20-mer sequence in purple. Transfection concentrations are indicated above the gel image. Relative abundance (%) of amplicons are shown in the graph below each gel image. GTC, Gene Tools control; Neg, no template PCR control; UT, untreated; bp, base pairs; FL, full-length amplicon; nM, nanomolar.

FIG. 10 provides evaluation of exon 3+4 SSO cocktails in Huh7 cells. RT-PCR analysis of MDK transcripts after Lipofectamine 3000 transfection with cocktails of one SSO targeting each MDK exon 3 and exon 4. Transfection concentrations are indicated above the gel image. Relative abundance (%) of amplicons are shown in the graph below each gel image. GTC, Gene Tools control; Neg, no template PCR control; UT, untreated; bp, base pairs; FL, full-length amplicon; nM, nanomolar; i, intron.

FIG. 11 provides evaluation of SSOs in SHSY5Y cells. RT-PCR analysis of MDK transcripts after Lipofectamine 3000 transfection with individual SSOs targeting MDK a) exon 3 and b) exon 4 or c) two-SSO cocktails. Transfection concentrations are indicated above the gel image. Relative abundance (%) of amplicons are shown in the graph beside each gel image. GTC, Gene Tools control; L3K, Lipofectamine 3000 transfection reagent; Neg, no template PCR control; UT, untreated; bp, base pairs; FL, full-length amplicon; nM, nanomolar; i, intron.

FIG. 12 provides evaluation of promising SSOs in HMC-1 cells. a) RT-PCR analysis of MDK transcripts after Lipofectamine 3000 transfection with the most promising SSO targeting MDK exon 3 and 4. Transfection concentrations are indicated above the gel image. b) The SMN positive control indicates transfection efficiency. Relative abundance (%) of amplicons are shown below each gel image. GTC, Gene Tools control; L3K, Lipofectamine 3000 transfection reagent control; Neg, no template PCR control; UT, untreated; bp, base pairs; FL, full-length amplicon; nM, nanomolar. c) Amplification of HMC-1 RNA treated with ezDNase or left untreated with no reverse transcription step toconfirm effective removal of contaminating gDNA.

FIG. 13 provides further evaluation of promising SSOs in HMC-1 cells. a) RT-PCR analysis of MDK transcripts after Lipofectamine 3000 transfection with the most promising SSOs targeting MDK exon 3 and 4. Transfection concentrations are indicated above the gel image. b) The SMN positive control indicates transfection efficiency. Relative abundance (%) of amplicons are shown below each gel image. GTC, Gene Tools control; L3K, Lipofectamine 3000 transfection reagent; Neg, no template PCR control; UT, untreated; bp, base pairs; FL, full-length amplicon; nM, nanomolar.

FIG. 14 provides evaluation of the promising exon 4 microwalked SSOs in HMC-1 cells. a) RT-PCR analysis of MDK transcripts after Lipofectamine 3000 transfection with the most promising SSOs microwalked around the original exon 4 sequences. Transfection concentrations are indicated above the gel image. b) The SMN positive control indicates transfection efficiency. Relative abundance (%) of amplicons are shown below each gel image. GTC, GeneTools control; Neg, no template PCR control; UT, untreated; bp, base pairs; FL, full-length amplicon; nM, nanomolar.

FIG. 15 provides an evaluation of the effects of skipping at the pre-mRNA level of Huh7 cells transfected with the most promising 2′OMe-PS SSOs targeting MDK exon 3 and 4 using Lipofectamine 3000. Transfection concentrations are indicated above the images. Relative abundance (%) of amplicons are shown in the graph below the RT-PCR gel image. GTC, Gene Tools control; L3K, Lipofectamine 3000 transfection reagent; Neg, no template PCR control; UT, untreated; bp, base pairs; FL, full-length amplicon; nM, nanomolar.

FIG. 16 provides an evaluation of the effects of skipping with antisense oligonucleotides synthesised using phosphorodiamidate morpholino (PMO) nucleotide chemistry targeting the most promising regions of MDK Exon 3 and 4 RT-PCR analysis of SHSY5Y cells transfected with one Exon 3 and three Exon 4 PMO SSOs using Neon transfection reagent. Transfection concentrations are indicated above the images. Relative abundance (%) of amplicons are shown in the graph below the RT-PCR gel image. GTC, Gene Tools control; Zap, Neon transfection reagent; UT, untreated; bp, base pairs; FL, full-length amplicon; nM, nanomolar.

FIG. 17 provides further evaluation of SSOs at the protein level. Western blot analysis of Huh7 cell a), SHSY5Y cells b), transfected with the most promising PMO AOs targeting MDK exon 3 and 4 using Lipofectamine 3000. Transfection concentrations are indicated above the images. GTC, Gene Tools control; L3K, lipofectamine 3000 transfection reagent.

### KEY TO THE SEQUENCE LISTING

SEQ ID NO:1 hMDK Ex1F
SEQ ID NO:2 hMDK Ex5R (o)
SEQ ID NO:3 hMDK Ex5R (i)
SEQ ID NO:4 hMDK Ex2F (o)
SEQ ID NO:5 hMDK Ex2F (i)
SEQ ID NO:6 Homo sapiens midkine full length sequence
SEQ ID NO:7 Homo sapiens midkine region 1
SEQ ID NO:8 Homo sapiens midkine region 2
SEQ ID NO:9 Homo sapiens midkine region 3
SEQ ID NO:10 Homo sapiens midkine region 4
SEQ ID NO:11 Homo sapiens midkine region 5
SEQ ID NO: 12 Homo sapiens midkine region 6
SEQ ID NO:13 Homo sapiens midkine region 7
SEQ ID NO: 14 Homo sapiens midkine region 8
SEQ ID NO: 15 Homo sapiens midkine region 9
SEQ ID NO: 16 Homo sapiens midkine region 10
SEQ ID NO: 17 Homo sapiens midkine region 11
SEQ ID NO: 18 Homo sapiens midkine region 12
SEQ ID NO: 19 Homo sapiens midkine region 13
SEQ ID NO:20 Homo sapiens midkine region 14
SEQ ID NO:21 Homo sapiens midkine region 15
SEQ ID NO:22 Homo sapiens midkine region 16
SEQ ID NO:23 Homo sapiens midkine region 17
SEQ ID NO:24 Homo sapiens midkine region 18
SEQ ID NO:25 Homo sapiens midkine region 19
SEQ ID NO:26 Homo sapiens midkine region 20
SEQ ID NO:27 Homo sapiens midkine region 21
SEQ ID NO:28 Homo sapiens midkine region 22
SEQ ID NO:29 Homo sapiens midkine region 23
SEQ ID NO:30 Homo sapiens midkine region 24
SEQ ID NO:31 Homo sapiens midkine region 25
SEQ ID NO:32 Homo sapiens midkine region 26
SEQ ID NO:33 Homo sapiens midkine region 27
SEQ ID NO:34 Homo sapiens midkine region 28
SEQ ID NO:35 Homo sapiens midkine region 29
SEQ ID NO:36 Homo sapiens midkine region 30
SEQ ID NO:37 Homo sapiens midkine region 31
SEQ ID NO:38 Homo sapiens midkine region 32
SEQ ID NO:39 Homo sapiens midkine region 33
SEQ ID NO:40 Homo sapiens midkine region 34
SEQ ID NO:41 Homo sapiens midkine region 35
SEQ ID NO:42 Homo sapiens midkine region 36
SEQ ID NO:43 MDK H3A(−12+13)
SEQ ID NO:44 MDK H3A(−04+21)
SEQ ID NO:45 MDK H3A(+89+113)
SEQ ID NO:46 MDK H3A(+143+167)
SEQ ID NO:47 MDK H3A(+146+165)
SEQ ID NO:48 MDK H3D(+15−10)
SEQ ID NO:49 MDK H4A(−12+13)
SEQ ID NO:50 MDK H4A(−04+21)
SEQ ID NO:51 MDK H4A(+74+98)
SEQ ID NO:52 MDK H4A(+106+130)
SEQ ID NO:53 MDK H4D(+10−15)
SEQ ID NO:54 MDK H3A(+133+157)
SEQ ID NO:55 MDK H3A(+138+162)
SEQ ID NO:56 MDK H3D(+21−04)
SEQ ID NO:57 MDK H3D(+10−15)
SEQ ID NO:58 MDK H3D(+05−20)
SEQ ID NO:59 MDK H4A(+69+93)
SEQ ID NO:60 MDK H4A(+79+103)
SEQ ID NO:61 MDK H4A(+101+125)
SEQ ID NO:62 MDK H4A(+111+135)
SEQ ID NO:63 MDK H4D(+15−10)
SEQ ID NO:64 MDK H4D(+05−20)
SEQ ID NO:65 MDK H4A(+100+124)

### DETAILED DESCRIPTION

### General Techniques and Definitions

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in cell culture, molecular biology, immunology, immunohistochemistry, protein chemistry, and biochemistry).

Unless otherwise indicated, the recombinant protein, cell culture, molecular biology, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

The words “a” and “an” when used in this disclosure, including the claims, denotes “one or more.”

As used herein, the terms “about” and “approximately” are interchangeable, and should generally be understood to refer to a range of numbers around a given number, as well as to all numbers in a recited range of numbers. Moreover, all numerical ranges herein should be understood to include each whole integer within the range. Unless stated to the contrary, the terms “about” and “approximately” refers to +/−10%, more preferably +/−5%, more preferably +/−1%, of the designated value.

The terms “e.g.,” and “i.e.” as used herein, is used merely by way of example, without limitation intended, and should not be construed as referring only those items explicitly enumerated in the disclosure.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Splice Switching Oligonucleotides

As described herein, the present disclosure provides splice-switching oligonucleotides (SSO) targeting the pre-mRNA sequence of human midkine. An SSO of the disclosure is between 10 and 50 nucleotides in length (e.g., between 20-25 nucleotides in length), and comprises a nucleotide sequence of at least 10 contiguous nucleotides which is substantially complementary to a target region of corresponding length within a pre-mRNA sequence of human midkine.

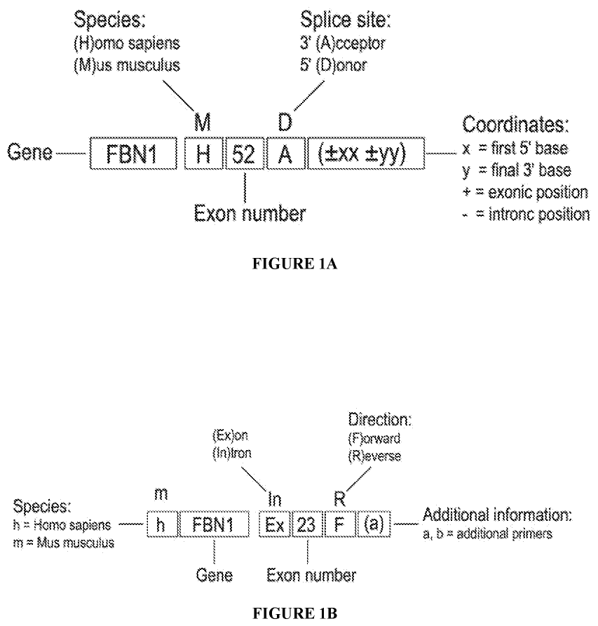

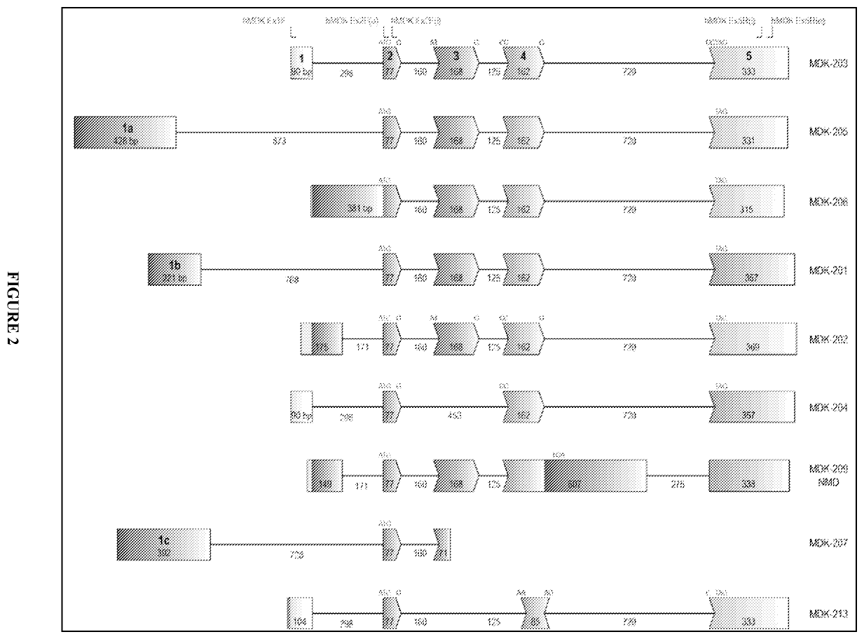

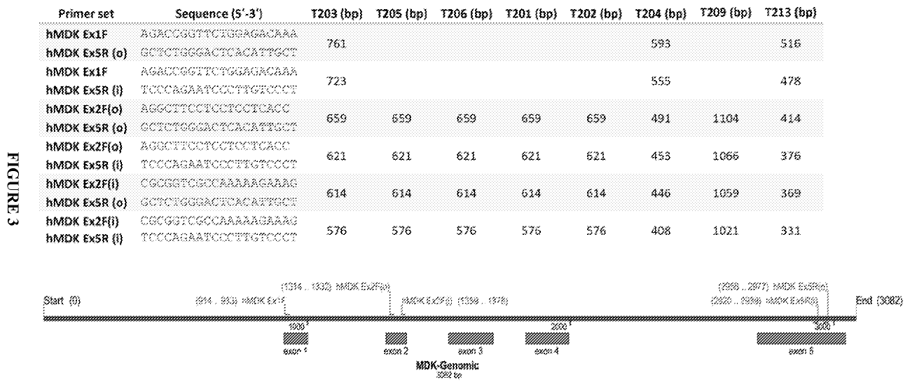

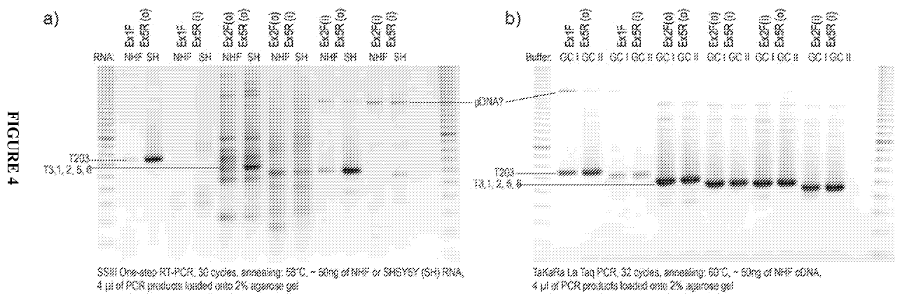

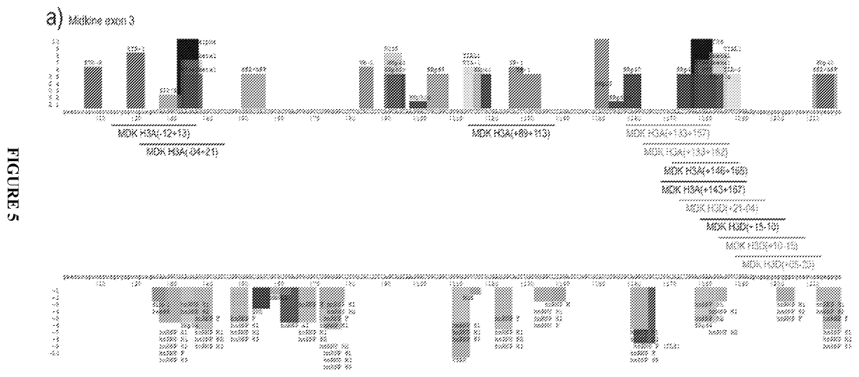

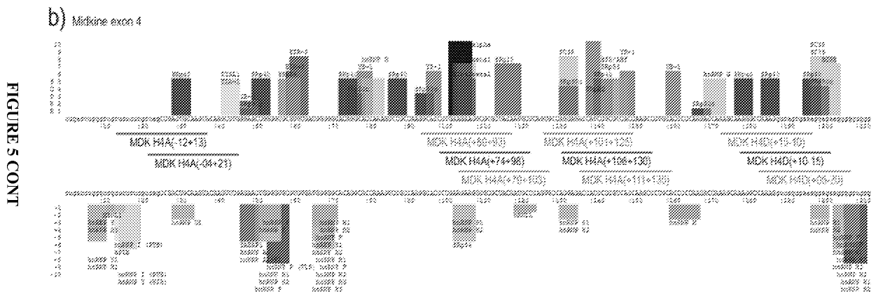

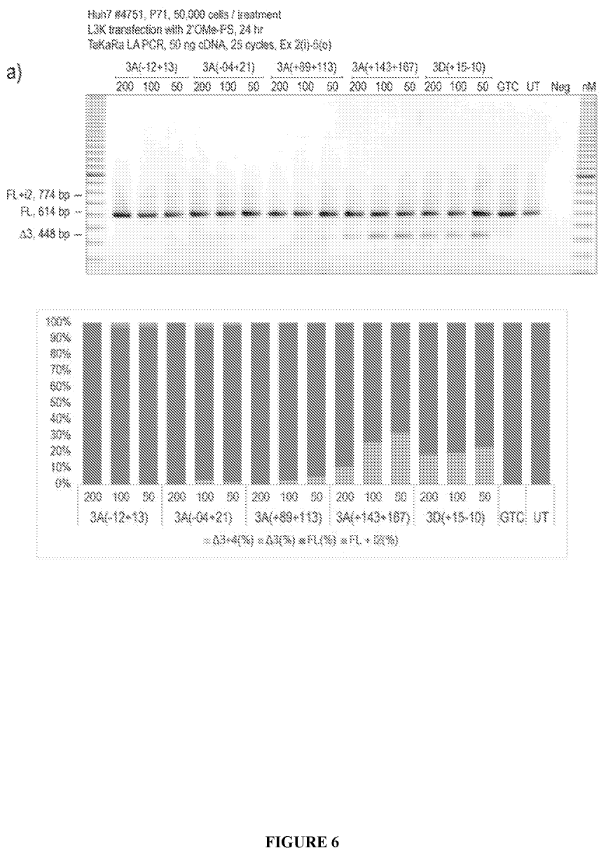

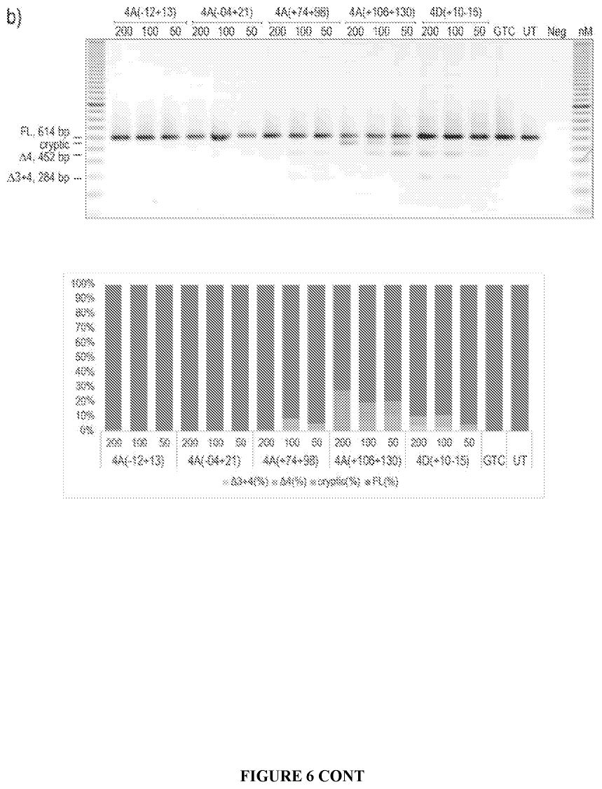

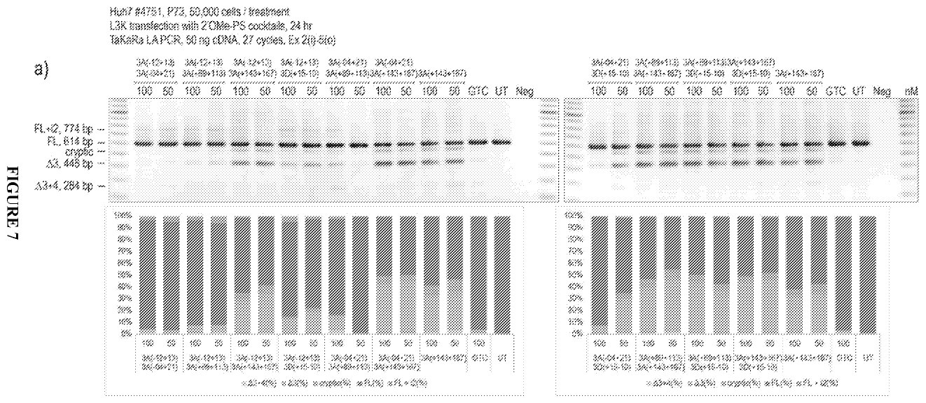

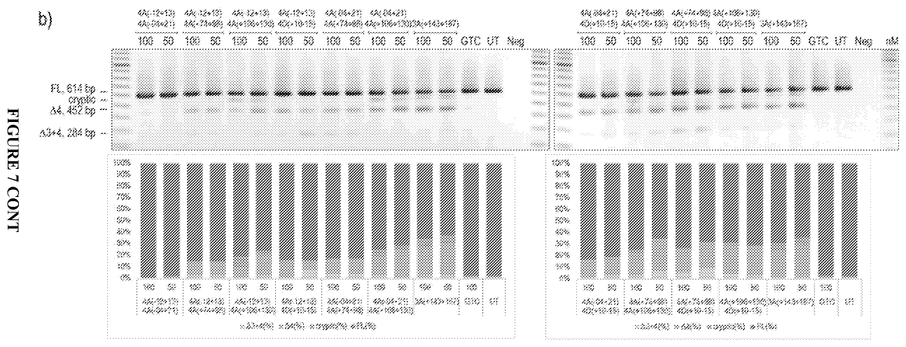

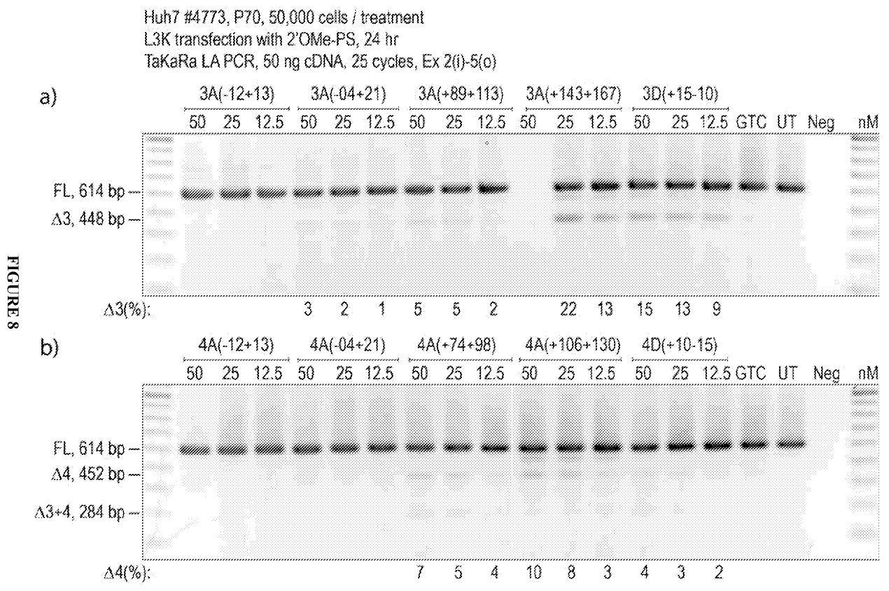

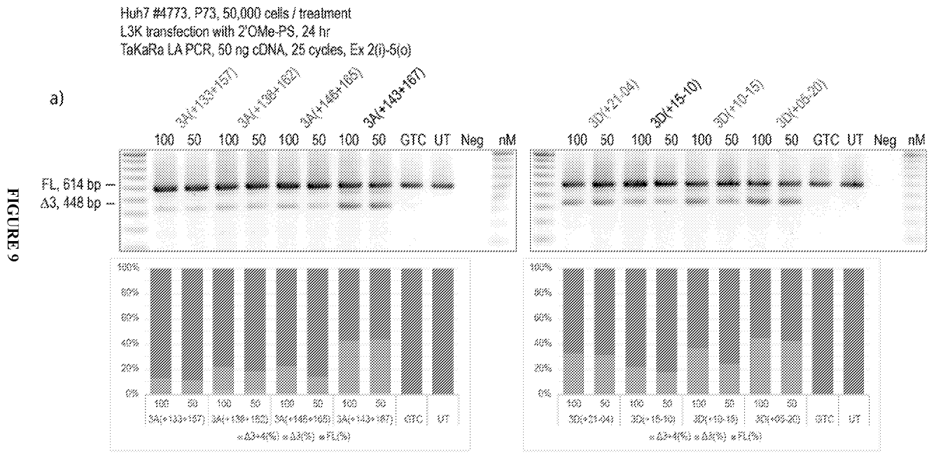

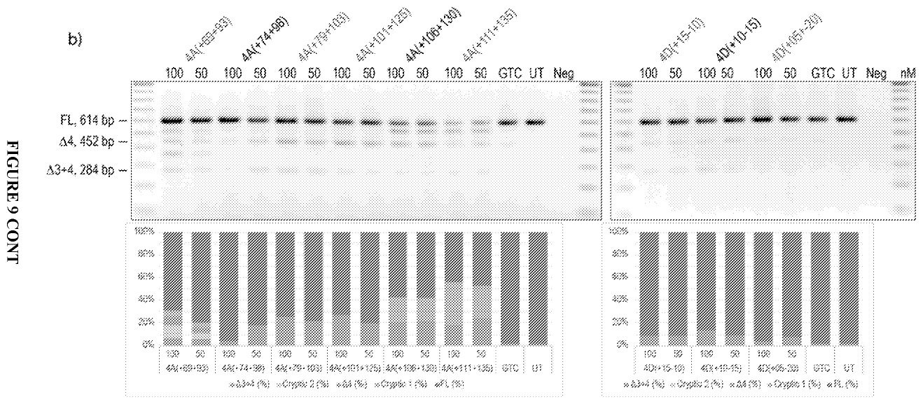

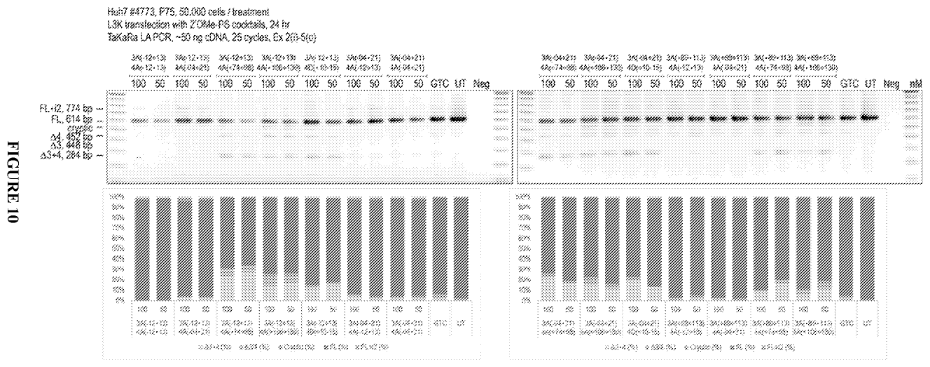

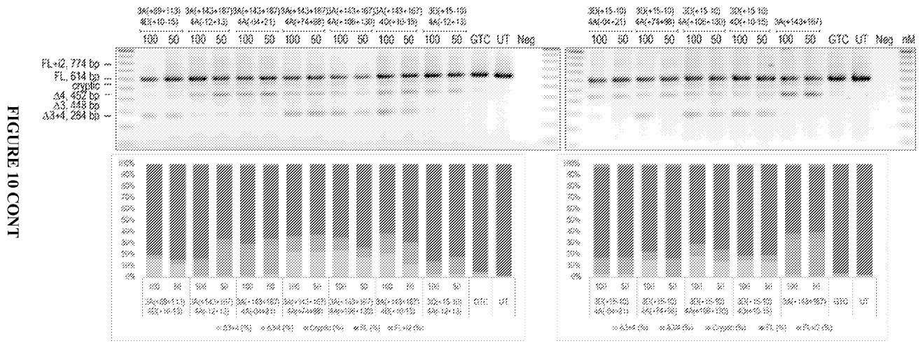

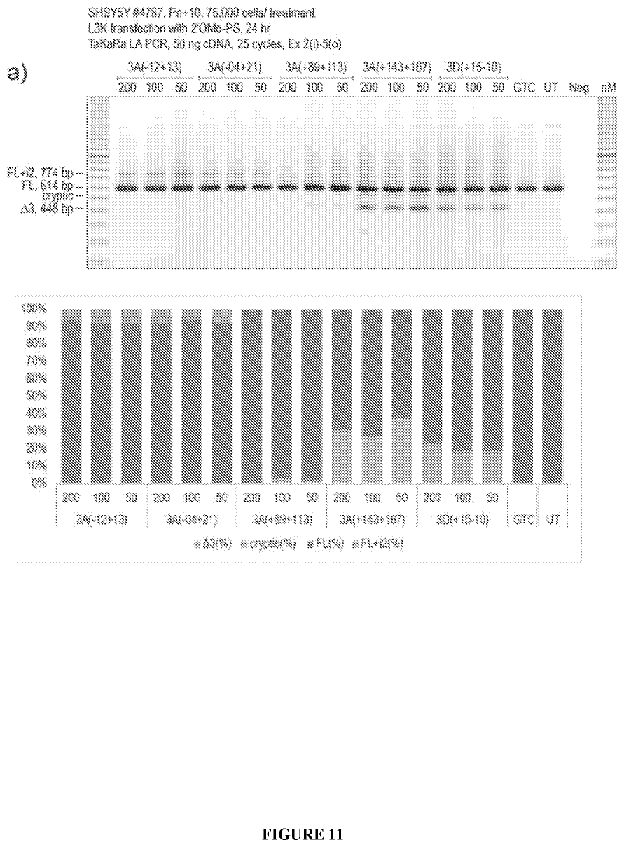

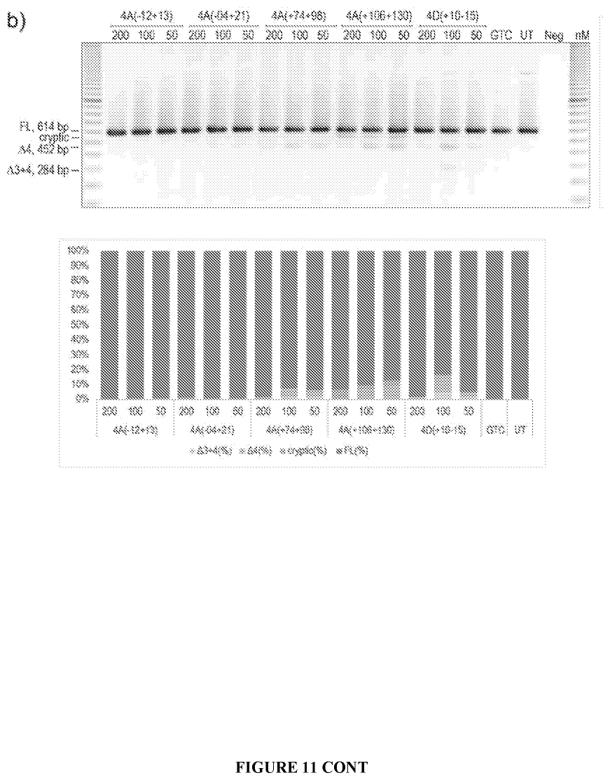

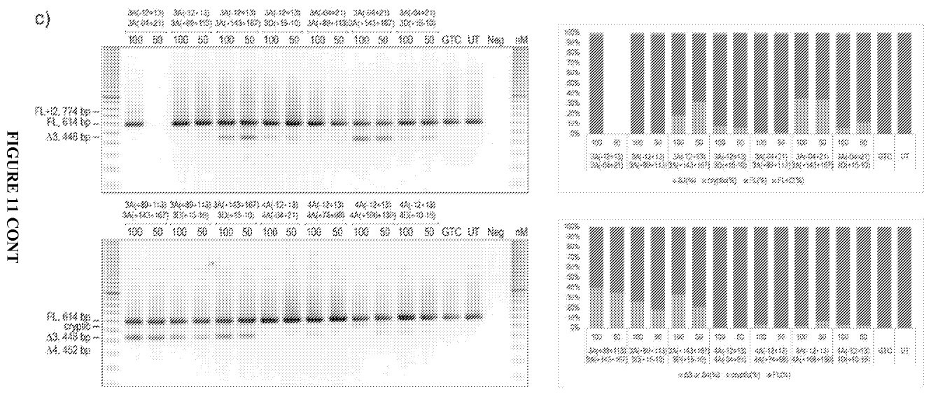

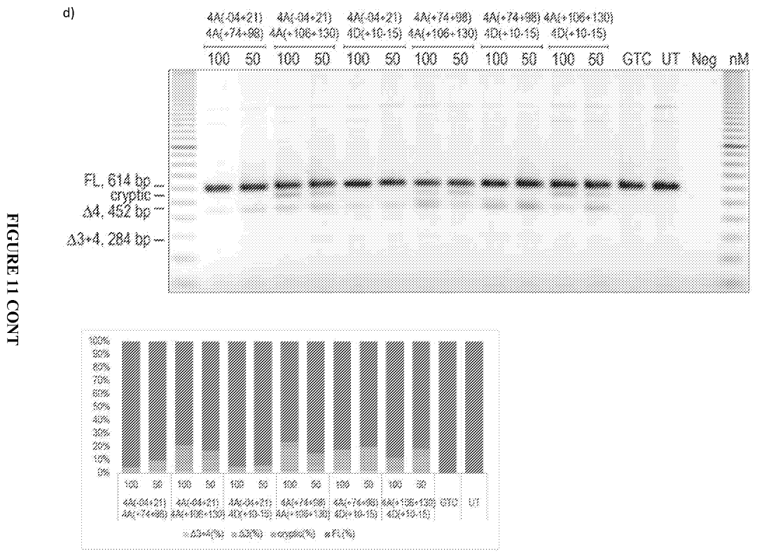

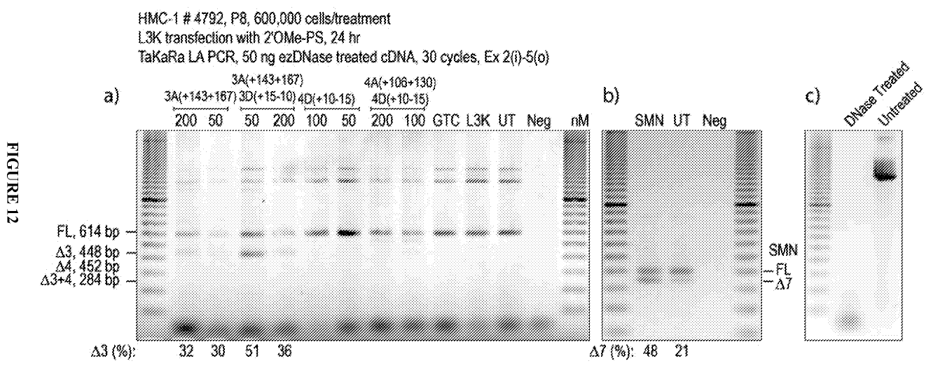

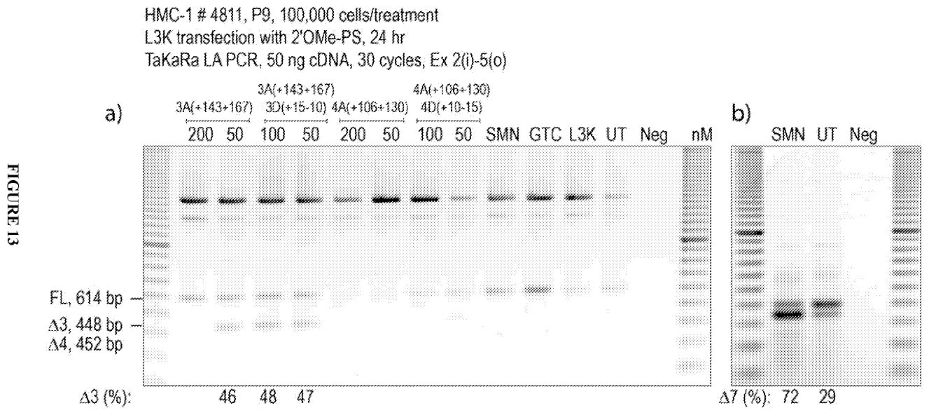

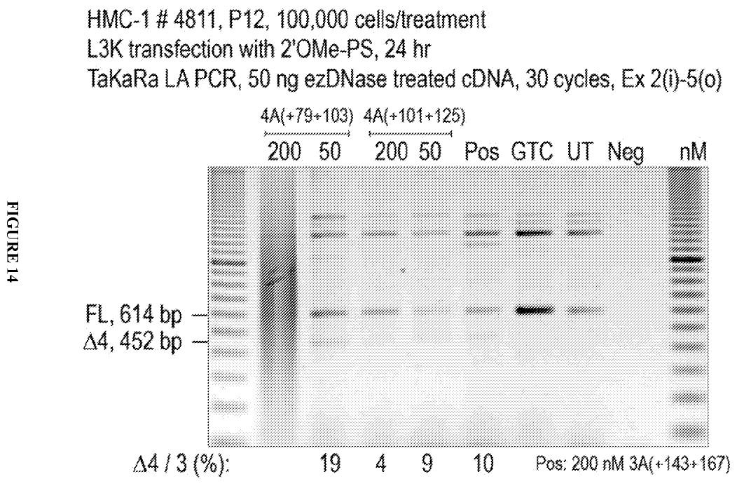

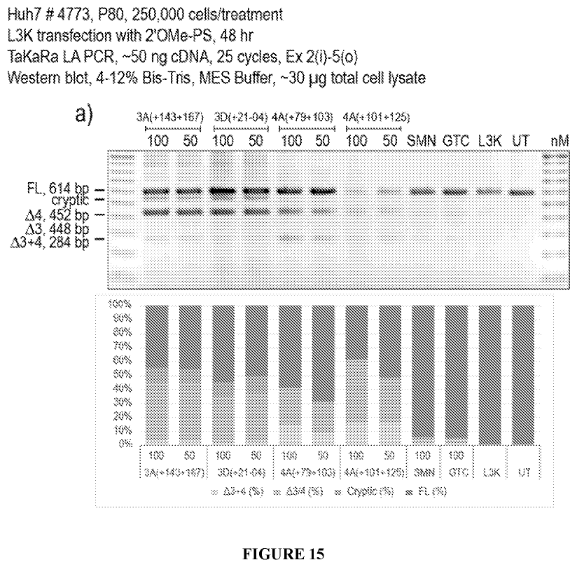

As used herein, the terms “splice-switching oligonucleotide”, “splice switching oligomer”, “SSO”, and “antisense oligonucleotide”, or “AO” when used in the context of splice switching, refers to a short oligonucleotide that is substantially complementary to, and able to base-pair with, a portion of a pre-mRNA molecule and thereby disrupt the normal splicing repertoire of the transcript by blocking the RNA-RNA base-pairing or protein-RNA binding interactions that occur between components of the splicing machinery and the pre-mRNA. In doing so, a splice-switching oligonucleotide is able to induce targeted exon skipping. The use of SSOs for targeted exon skipping is well known in the art [see for example, G. Hartmann and S. Endres, Manual of Antisense Methodology, Kluwer (1999)]. AO nomenclature system was proposed and published (Aung-Htut M T et al 2019 Int J Mol Sci 20:5030) to distinguish between the different antisense molecules (see FIG. 1).

The term “oligonucleotide” refers to an oligomer or polymer of ribonucleic acid (RNA) or deoxyribonucleic acid (DNA), wherein the polymer or oligomer of nucleotide monomers contains any combination of nucleotides or nucleosides, modified nucleotides or nucleosides, sugars, modified sugars, phosphate bridges, or modified phosphorus atom bridges (also referred to herein as “inter-nucleotidic linkage”). Oligonucleotides can be single-stranded or double-stranded or a combination thereof. A single-stranded oligonucleotide can have double-stranded regions and a double-stranded oligonucleotide can have single-stranded regions (such as a microRNA or shRNA).

“RNA” as described herein is meant as a molecule comprising at least one ribonucleotide residue. By “ribonucleotide” is meant a nucleotide with a hydroxyl group at the 2′ position of a β-D-ribo-furanose moiety. The terms include double-stranded RNA, single-stranded RNA, isolated RNA, such as, messenger RNA as well as altered RNA that differs from naturally occurring RNA by the addition, deletion, substitution and/or alteration of one or more nucleotides. Nucleotides in the RNA molecules of the instant disclosure can also comprise non-standard nucleotides, such as non-naturally occurring nucleotides or chemically synthesized nucleotides or deoxynucleotides.

The SSO and the DNA or RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or precise pairing such that stable and specific binding occurs between the oligonucleotide and the DNA or RNA target. It is understood in the art that the sequence of an SSO need not be 100% complementary to that of its target region to be specifically hybridisable. An SSO is specifically hybridisable when binding of the compound to the target DNA or RNA molecule interferes with the normal function of the target DNA or RNA, and there is a sufficient degree of complementarity to avoid non-specific binding of the SSO to non-target regions under conditions in which specific binding is desired, i.e., under physiological conditions in the case of in vivo assays or therapeutic treatment, and in the case of in vitro assays, under conditions in which the assays are performed.

As used herein, the term “complementary” with regard to a sequence refers to a complement of the sequence by Watson-Crick base pairing, whereby guanine (G) pairs with cytosine (C), and adenine (A) pairs with either uracil (U) or thymine (T). A sequence may be complementary to the entire length of another sequence, or it may be complementary to a specified portion or length of another sequence. One of skill in the art will recognize that U may be present in RNA, and that T may be present in DNA. Therefore, an A within either of a RNA or DNA sequence may pair with a U in a RNA sequence or T in a DNA sequence.

As used herein, the term “substantially complementary” is used to indicate a sufficient degree of complementarity or precise pairing such that stable and specific binding occurs between nucleic acid sequences e.g., between the SSO and the SSO complement sequence or between the SSO and the target region. That is, the SSO is able to “specifically hybridise” to its cognate target region. It is understood that the sequence of a nucleic acid need not be 100% complementary to that of its target or complement. The term encompasses a sequence complementary to another sequence with the exception of an overhang. In some cases, the sequence is complementary to the other sequence with the exception of 1 to 4 mismatches. In some cases, the sequences are complementary except for 1 mismatch. In some cases, the sequences are complementary except for 2 mismatches. In other cases, the sequences are complementary except for 3 mismatches. In yet other cases, the sequences are complementary except for 4 mismatches.

The SSO may be capable of hybridising to its target region under physiological conditions i.e., under normal conditions in a cell.

As used herein, a “target” or “target region” refers to a stretch of nucleotides within a pre-mRNA sequence of human midkine to which an SSO of the disclosure is substantially complementary (or complementary) and able to hybridise (e.g., under physiological conditions).

The length of an SSO of the disclosure may vary so long as it is capable of binding selectively to the intended location within the pre-mRNA molecule. In this regard, it is well known by those skilled in the art that it is possible to increase or decrease the length of a SSO and/or introduce mismatch bases without eliminating activity. Methods to determine desired activity are disclosed herein and well known to those skilled in the art.

Generally, the SSO will comprise a nucleotide sequence from about 10 nucleotides in length up to about 50 nucleotides in length. For example, the SSO may be about 10 nucleotides in length, or about 15 nucleotides in length, or about 20 nucleotides in length, or about 25 nucleotides in length, or about 30 nucleotides in length, or about 35 nucleotides in length, or about 40 nucleotides in length, or about 45 nucleotides in length, or about 50 nucleotides in length. In particular examples, the length of the SSO is between 15 to 30 nucleotides in length, such as 15 to 25 nucleotides in length.

SSOs of the disclosure may be designed to disrupt splicing and thereby induce exon skipping of exon 1, 2, 3, 4 or 5 of human midkine.

As used herein “exon skipping” refers to altering the processing of a pre-mRNA transcript such that the spliced mRNA molecule contains a different combination of exons as a result of exon skipping. In the context of the present disclosure, exon skipping refers to altering splicing of MDK pre-mRNA to achieve a deletion of one or more exons.

In one example, an SSO of the disclosure is designed to specifically hybridise to a target region of corresponding length within the pre-mRNA sequence of human midkine to disrupt splicing and thereby induce exon skipping of exon 3. The degree of identity of the SSO nucleotide sequence to the target region should be at least 85%, 90%, 95% or 100%. The SSO may of course comprise unrelated sequences which may function to stabilize the molecule such as described herein.

In accordance with this example, the SSO may comprise a nucleotide sequence of at least 10 contiguous nucleotides (e.g., 10, or 11, or 12, or 13, or 14, or 15, or 16, or 17, or 18, or 19, or 20, or 21, or 22, or 23, or 24, or 25 contiguous nucleotides) which is substantially complementary to a region of corresponding length in a sequence set forth in any one of SEQ ID NOs: 7-14, 20-25 and 31-35. For example, the SSO may comprise a nucleotide sequence of at least 10 contiguous nucleotides (e.g., 10, or 11, or 12, or 13, or 14, or 15, or 16, or 17, or 18, o 19, or 20, or 21, or 22, or 23, or 24, or 25 contiguous nucleotides) which is complementary to a region of corresponding length in a sequence set forth in any one of SEQ ID NOs: 7-14, 20-25 and 31-35, optionally with the exception of 1, 2, 3 or 4 mismatches.

For example, the SSO may comprise a nucleotide sequence which is substantially complementary to SEQ ID NO: 7. For example, the SSO may comprise a nucleotide sequence which is complementary to SEQ ID NO: 7 with the exception of 4 base pair mismatches. For example, the SSO may comprise a nucleotide sequence which is complementary to SEQ ID NO: 7 with the exception of 3 base pair mismatches. For example, the SSO may comprise a nucleotide sequence which is complementary to SEQ ID NO: 7 with the exception of 2 base pair mismatches. For example, the SSO may comprise a nucleotide sequence which is complementary to SEQ ID NO: 7 with the exception of 1 base pair mismatch. In a particular example, the SSO may comprise a nucleotide sequence which is 100% complementary to SEQ ID NO: 7.

For example, the SSO may comprise a nucleotide sequence which is substantially complementary to SEQ ID NO: 8. For example, the SSO may comprise a nucleotide sequence which is complementary to SEQ ID NO: 8 with the exception of 4 base pair mismatches. For example, the SSO may comprise a nucleotide sequence which is complementary to SEQ ID NO: 8 with the exception of 3 base pair mismatches. For example, the SSO may comprise a nucleotide sequence which is complementary to SEQ ID NO: 8 with the exception of 2 base pair mismatches. For example, the SSO may comprise a nucleotide sequence which is complementary to SEQ ID NO: 8 with the exception of 1 base pair mismatch. In a particular example, the SSO may comprise a nucleotide sequence which is 100% complementary SEQ ID NO: 8.

For example, the SSO may comprise a nucleotide sequence which is substantially complementary to SEQ ID NO: 9. For example, the SSO may comprise a nucleotide sequence which is complementary to SEQ ID NO: 9 with the exception of 4 base pair mismatches. For example, the SSO may comprise a nucleotide sequence which is complementary to SEQ ID NO: 9 with the exception of 3 base pair mismatches. For example, the SSO may comprise a nucleotide sequence which is complementary to SEQ ID NO: 9 with the exception of 2 base pair mismatches. For example, the SSO may comprise a nucleotide sequence which is complementary to SEQ ID NO: 9 with the exception of 1 base pair mismatch. In a particular example, the SSO may comprise a nucleotide sequence which is 100% complementary SEQ ID NO: 9.

For example, the SSO may comprise a nucleotide sequence which is substantially complementary to SEQ ID NO: 10. For example, the SSO may comprise a nucleotide sequence which is complementary to SEQ ID NO: 10 with the exception of 4 base pair mismatches. For example, the SSO may comprise a nucleotide sequence which is complementary to SEQ ID NO: 10 with the exception of 3 base pair mismatches. For example, the SSO may comprise a nucleotide sequence which is complementary to SEQ ID NO: 10 with the exception of 2 base pair mismatches. For example, the SSO may comprise a nucleotide sequence which is complementary to SEQ ID NO: 10 with the exception of 1 base pair mismatch. In a particular example, the SSO may comprise a nucleotide sequence which is 100% complementary SEQ ID NO: 10.

For example, the SSO may comprise a nucleotide sequence which is substantially complementary to SEQ ID NO: 11. For example, the SSO may comprise a nucleotide sequence which is complementary to SEQ ID NO: 11 with the exception of 4 base pair mismatches. For example, the SSO may comprise a nucleotide sequence which is complementary to SEQ ID NO: 11 with the exception of 3 base pair mismatches. For example, the SSO may comprise a nucleotide sequence which is complementary to SEQ ID NO: 11 with the exception of 2 base pair mismatches. For example, the SSO may comprise a nucleotide sequence which is complementary to SEQ ID NO: 11 with the exception of 1 base pair mismatch. In a particular example, the SSO may comprise a nucleotide sequence which is 100% complementary SEQ ID NO: 11.

For example, the SSO may comprise a nucleotide sequence which is substantially complementary to SEQ ID NO: 12. For example, the SSO may comprise a nucleotide sequence which is complementary to SEQ ID NO: 12 with the exception of 4 base pair mismatches. For example, the SSO may comprise a nucleotide sequence which is complementary to SEQ ID NO: 12 with the exception of 3 base pair mismatches. For example, the SSO may comprise a nucleotide sequence which is complementary to SEQ ID NO: 12 with the exception of 2 base pair mismatches. For example, the SSO may comprise a nucleotide sequence which is complementary to SEQ ID NO: 12 with the exception of 1 base pair mismatch. In a particular example, the SSO may comprise a nucleotide sequence which is 100% complementary SEQ ID NO: 12.

For example, the SSO may comprise a nucleotide sequence which is substantially complementary to SEQ ID NO: 13. For example, the SSO may comprise a nucleotide sequence which is complementary to SEQ ID NO: 13 with the exception of 4 base pair mismatches. For example, the SSO may comprise a nucleotide sequence which is complementary to SEQ ID NO: 13 with the exception of 3 base pair mismatches. For example, the SSO may comprise a nucleotide sequence which is complementary to SEQ ID NO: 13 with the exception of 2 base pair mismatches. For example, the SSO may comprise a nucleotide sequence which is complementary to SEQ ID NO: 13 with the exception of 1 base pair mismatch. In a particular example, the SSO may comprise a nucleotide sequence which is 100% complementary SEQ ID NO: 13.

For example, the SSO may comprise a nucleotide sequence which is substantially complementary to SEQ ID NO: 14. For example, the SSO may comprise a nucleotide sequence which is complementary to SEQ ID NO: 14 with the exception of 4 base pair mismatches. For example, the SSO may comprise a nucleotide sequence which is complementary to SEQ ID NO: 14 with the exception of 3 base pair mismatches. For example, the SSO may comprise a nucleotide sequence which is complementary to SEQ ID NO: 14 with the exception of 2 base pair mismatches. For example, the SSO may comprise a nucleotide sequence which is complementary to SEQ ID NO: 14 with the exception of 1 base pair mismatch. In a particular example, the SSO may comprise a nucleotide sequence which is 100% complementary SEQ ID NO: 14.

For example, the SSO may comprise a nucleotide sequence which is substantially complementary to SEQ ID NO: 20. For example, the SSO may comprise a nucleotide sequence which is complementary to SEQ ID NO: 20 with the exception of 4 base pair mismatches. For example, the SSO may comprise a nucleotide sequence which is complementary to SEQ ID NO: 20 with the exception of 3 base pair mismatches. For example, the SSO may comprise a nucleotide sequence which is complementary to SEQ ID NO: 20 with the exception of 2 base pair mismatches. For example, the SSO may comprise a nucleotide sequence which is complementary to SEQ ID NO: 20 with the exception of 1 base pair mismatch. In a particular example, the SSO may comprise a nucleotide sequence which is 100% complementary SEQ ID NO: 20.

For example, the SSO may comprise a nucleotide sequence which is substantially complementary to SEQ ID NO: 21. For example, the SSO may comprise a nucleotide sequence which is complementary to SEQ ID NO: 21 with the exception of 4 base pair mismatches. For example, the SSO may comprise a nucleotide sequence which is complementary to SEQ ID NO: 21 with the exception of 3 base pair mismatches. For example, the SSO may comprise a nucleotide sequence which is complementary to SEQ ID NO: 21 with the exception of 2 base pair mismatches. For example, the SSO may comprise a nucleotide sequence which is complementary to SEQ ID NO: 21 with the exception of 1 base pair mismatch. In a particular example, the SSO may comprise a nucleotide sequence which is 100% complementary SEQ ID NO: 21.

For example, the SSO may comprise a nucleotide sequence which is substantially complementary to SEQ ID NO: 22. For example, the SSO may comprise a nucleotide sequence which is complementary to SEQ ID NO: 22 with the exception of 4 base pair mismatches. For example, the SSO may comprise a nucleotide sequence which is complementary to SEQ ID NO: 22 with the exception of 3 base pair mismatches. For example, the SSO may comprise a nucleotide sequence which is complementary to SEQ ID NO: 22 with the exception of 2 base pair mismatches. For example, the SSO may comprise a nucleotide sequence which is complementary to SEQ ID NO: 22 with the exception of 1 base pair mismatch. In a particular example, the SSO may comprise a nucleotide sequence which is 100% complementary SEQ ID NO: 22.

For example, the SSO may comprise a nucleotide sequence which is substantially complementary to SEQ ID NO: 23. For example, the SSO may comprise a nucleotide sequence which is complementary to SEQ ID NO: 23 with the exception of 4 base pair mismatches. For example, the SSO may comprise a nucleotide sequence which is complementary to SEQ ID NO: 23 with the exception of 3 base pair mismatches. For example, the SSO may comprise a nucleotide sequence which is complementary to SEQ ID NO: 23 with the exception of 2 base pair mismatches. For example, the SSO may comprise a nucleotide sequence which is complementary to SEQ ID NO: 23 with the exception of 1 base pair mismatch. In a particular example, the SSO may comprise a nucleotide sequence which is 100% complementary SEQ ID NO: 23.

For example, the SSO may comprise a nucleotide sequence which is substantially complementary to SEQ ID NO: 24. For example, the SSO may comprise a nucleotide sequence which is complementary to SEQ ID NO: 24 with the exception of 4 base pair mismatches. For example, the SSO may comprise a nucleotide sequence which is complementary to SEQ ID NO: 24 with the exception of 3 base pair mismatches. For example, the SSO may comprise a nucleotide sequence which is complementary to SEQ ID NO: 24 with the exception of 2 base pair mismatches. For example, the SSO may comprise a nucleotide sequence which is complementary to SEQ ID NO: 24 with the exception of 1 base pair mismatch. In a particular example, the SSO may comprise a nucleotide sequence which is 100% complementary SEQ ID NO: 24.

For example, the SSO may comprise a nucleotide sequence which is substantially complementary to SEQ ID NO: 25. For example, the SSO may comprise a nucleotide sequence which is complementary to SEQ ID NO: 25 with the exception of 4 base pair mismatches. For example, the SSO may comprise a nucleotide sequence which is complementary to SEQ ID NO: 25 with the exception of 3 base pair mismatches. For example, the SSO may comprise a nucleotide sequence which is complementary to SEQ ID NO: 25 with the exception of 2 base pair mismatches. For example, the SSO may comprise a nucleotide sequence which is complementary to SEQ ID NO: 25 with the exception of 1 base pair mismatch. In a particular example, the SSO may comprise a nucleotide sequence which is 100% complementary SEQ ID NO: 25.

For example, the SSO may comprise a nucleotide sequence which is substantially complementary to SEQ ID NO: 31. For example, the SSO may comprise a nucleotide sequence which is complementary to SEQ ID NO: 31 with the exception of 4 base pair mismatches. For example, the SSO may comprise a nucleotide sequence which is complementary to SEQ ID NO: 31 with the exception of 3 base pair mismatches. For example, the SSO may comprise a nucleotide sequence which is complementary to SEQ ID NO: 31 with the exception of 2 base pair mismatches. For example, the SSO may comprise a nucleotide sequence which is complementary to SEQ ID NO: 31 with the exception of 1 base pair mismatch. In a particular example, the SSO may comprise a nucleotide sequence which is 100% complementary SEQ ID NO: 31.

For example, the SSO may comprise a nucleotide sequence which is substantially complementary to SEQ ID NO: 32. For example, the SSO may comprise a nucleotide sequence which is complementary to SEQ ID NO: 32 with the exception of 4 base pair mismatches. For example, the SSO may comprise a nucleotide sequence which is complementary to SEQ ID NO: 32 with the exception of 3 base pair mismatches. For example, the SSO may comprise a nucleotide sequence which is complementary to SEQ ID NO: 32 with the exception of 2 base pair mismatches. For example, the SSO may comprise a nucleotide sequence which is complementary to SEQ ID NO: 32 with the exception of 1 base pair mismatch. In a particular example, the SSO may comprise a nucleotide sequence which is 100% complementary SEQ ID NO: 32.

For example, the SSO may comprise a nucleotide sequence which is substantially complementary to SEQ ID NO: 33. For example, the SSO may comprise a nucleotide sequence which is complementary to SEQ ID NO: 33 with the exception of 4 base pair mismatches. For example, the SSO may comprise a nucleotide sequence which is complementary to SEQ ID NO: 33 with the exception of 3 base pair mismatches. For example, the SSO may comprise a nucleotide sequence which is complementary to SEQ ID NO: 33 with the exception of 2 base pair mismatches. For example, the SSO may comprise a nucleotide sequence which is complementary to SEQ ID NO: 33 with the exception of 1 base pair mismatch. In a particular example, the SSO may comprise a nucleotide sequence which is 100% complementary SEQ ID NO: 33.

For example, the SSO may comprise a nucleotide sequence which is substantially complementary to SEQ ID NO: 34. For example, the SSO may comprise a nucleotide sequence which is complementary to SEQ ID NO: 34 with the exception of 4 base pair mismatches. For example, the SSO may comprise a nucleotide sequence which is complementary to SEQ ID NO: 34 with the exception of 3 base pair mismatches. For example, the SSO may comprise a nucleotide sequence which is complementary to SEQ ID NO: 34 with the exception of 2 base pair mismatches. For example, the SSO may comprise a nucleotide sequence which is complementary to SEQ ID NO: 34 with the exception of 1 base pair mismatch. In a particular example, the SSO may comprise a nucleotide sequence which is 100% complementary SEQ ID NO: 34.

For example, the SSO may comprise a nucleotide sequence which is substantially complementary to SEQ ID NO: 35. For example, the SSO may comprise a nucleotide sequence which is complementary to SEQ ID NO: 35 with the exception of 4 base pair mismatches. For example, the SSO may comprise a nucleotide sequence which is complementary to SEQ ID NO: 35 with the exception of 3 base pair mismatches. For example, the SSO may comprise a nucleotide sequence which is complementary to SEQ ID NO: 35 with the exception of 2 base pair mismatches. For example, the SSO may comprise a nucleotide sequence which is complementary to SEQ ID NO: 35 with the exception of 1 base pair mismatch. In a particular example, the SSO may comprise a nucleotide sequence which is 100% complementary SEQ ID NO: 35.

In one example, the SSO configured to induce skipping of exon 3 comprises a nucleotide sequence selected from the group of sequences set forth in SEQ ID NO: 43-48 and 54-58. In one particular example, the SSO configured to induce exon skipping of exon 3 comprises the sequence set forth in SEQ ID NO: 46.

For example, the SSO may comprise a nucleotide sequence which is substantially complementary to SEQ ID NO: 43. For example, the SSO may comprise a nucleotide sequence which is 85% complementary to SEQ ID NO: 43. For example, the SSO may comprise a nucleotide sequence which is 90% complementary to SEQ ID NO: 43. For example, the SSO may comprise a nucleotide sequence which is 95% complementary to SEQ ID NO: 43. In a particular example, the SSO may comprise a nucleotide sequence which is 100% complementary SEQ ID NO: 43.

For example, the SSO may comprise a nucleotide sequence which is substantially complementary to SEQ ID NO: 44. For example, the SSO may comprise a nucleotide sequence which is 85% complementary to SEQ ID NO: 44. For example, the SSO may comprise a nucleotide sequence which is 90% complementary to SEQ ID NO: 44. For example, the SSO may comprise a nucleotide sequence which is 95% complementary to SEQ ID NO: 44. In a particular example, the SSO may comprise a nucleotide sequence which is 100% complementary SEQ ID NO: 44.

For example, the SSO may comprise a nucleotide sequence which is substantially complementary to SEQ ID NO: 45. For example, the SSO may comprise a nucleotide sequence which is 85% complementary to SEQ ID NO: 45. For example, the SSO may comprise a nucleotide sequence which is 90% complementary to SEQ ID NO: 45. For example, the SSO may comprise a nucleotide sequence which is 95% complementary to SEQ ID NO: 45. In a particular example, the SSO may comprise a nucleotide sequence which is 100% complementary SEQ ID NO: 45.

For example, the SSO may comprise a nucleotide sequence which is substantially complementary to SEQ ID NO: 46. For example, the SSO may comprise a nucleotide sequence which is 85% complementary to SEQ ID NO: 46. For example, the SSO may comprise a nucleotide sequence which is 90% complementary to SEQ ID NO: 46. For example, the SSO may comprise a nucleotide sequence which is 95% complementary to SEQ ID NO: 46. In a particular example, the SSO may comprise a nucleotide sequence which is 100% complementary SEQ ID NO: 46.

For example, the SSO may comprise a nucleotide sequence which is substantially complementary to SEQ ID NO: 47. For example, the SSO may comprise a nucleotide sequence which is 85% complementary to SEQ ID NO: 47. For example, the SSO may comprise a nucleotide sequence which is 90% complementary to SEQ ID NO: 47. For example, the SSO may comprise a nucleotide sequence which is 95% complementary to SEQ ID NO: 47. In a particular example, the SSO may comprise a nucleotide sequence which is 100% complementary SEQ ID NO: 47.

For example, the SSO may comprise a nucleotide sequence which is substantially complementary to SEQ ID NO: 48. For example, the SSO may comprise a nucleotide sequence which is 85% complementary to SEQ ID NO: 48. For example, the SSO may comprise a nucleotide sequence which is 90% complementary to SEQ ID NO: 48. For example, the SSO may comprise a nucleotide sequence which is 95% complementary to SEQ ID NO: 48. In a particular example, the SSO may comprise a nucleotide sequence which is 100% complementary SEQ ID NO: 48.

For example, the SSO may comprise a nucleotide sequence which is substantially complementary to SEQ ID NO: 54. For example, the SSO may comprise a nucleotide sequence which is 85% complementary to SEQ ID NO: 54. For example, the SSO may comprise a nucleotide sequence which is 90% complementary to SEQ ID NO: 54. For example, the SSO may comprise a nucleotide sequence which is 95% complementary to SEQ ID NO: 54. In a particular example, the SSO may comprise a nucleotide sequence which is 100% complementary SEQ ID NO: 54.

For example, the SSO may comprise a nucleotide sequence which is substantially complementary to SEQ ID NO: 55. For example, the SSO may comprise a nucleotide sequence which is 85% complementary to SEQ ID NO: 55. For example, the SSO may comprise a nucleotide sequence which is 90% complementary to SEQ ID NO: 55. For example, the SSO may comprise a nucleotide sequence which is 95% complementary to SEQ ID NO: 55. In a particular example, the SSO may comprise a nucleotide sequence which is 100% complementary SEQ ID NO: 55.

For example, the SSO may comprise a nucleotide sequence which is substantially complementary to SEQ ID NO: 56. For example, the SSO may comprise a nucleotide sequence which is 85% complementary to SEQ ID NO: 56. For example, the SSO may comprise a nucleotide sequence which is 90% complementary to SEQ ID NO: 56. For example, the SSO may comprise a nucleotide sequence which is 95% complementary to SEQ ID NO: 56. In a particular example, the SSO may comprise a nucleotide sequence which is 100% complementary SEQ ID NO: 56.

For example, the SSO may comprise a nucleotide sequence which is substantially complementary to SEQ ID NO: 57. For example, the SSO may comprise a nucleotide sequence which is 85% complementary to SEQ ID NO: 57. For example, the SSO may comprise a nucleotide sequence which is 90% complementary to SEQ ID NO: 57. For example, the SSO may comprise a nucleotide sequence which is 95% complementary to SEQ ID NO: 57. In a particular example, the SSO may comprise a nucleotide sequence which is 100% complementary SEQ ID NO: 57.

For example, the SSO may comprise a nucleotide sequence which is substantially complementary to SEQ ID NO: 58. For example, the SSO may comprise a nucleotide sequence which is 85% complementary to SEQ ID NO: 58. For example, the SSO may comprise a nucleotide sequence which is 90% complementary to SEQ ID NO: 58. For example, the SSO may comprise a nucleotide sequence which is 95% complementary to SEQ ID NO: 58. In a particular example, the SSO may comprise a nucleotide sequence which is 100% complementary SEQ ID NO: 58.

In one example, an SSO of the disclosure is designed to specifically hybridise to a target region of corresponding length within the pre-mRNA sequence of human midkine to disrupt splicing and thereby induce exon skipping of exon 4. In accordance with this example, the SSO may comprise a nucleotide sequence of at least 10 contiguous nucleotides (e.g., 10, or 11, or 12, or 13, or 14, or 15, or 16, or 17, or 18, or 19, or 20, or 21, or 22, or 23, or 24, or 25 contiguous nucleotides) which is substantially complementary to a region of corresponding length in a sequence set forth in any one of SEQ ID NOs: 15-19, 26-30 and 36-42. For example, the SSO may comprise a nucleotide sequence of at least 10 contiguous nucleotides (e.g., 10, or 11, or 12, or 13, or 14, or 15, or 16, or 17, or 18, or 19, or 20, or 21, or 22, or 23, or 24, or 25 contiguous nucleotides) which is complementary to a region of corresponding length in a sequence set forth in any one of SEQ ID NOs: 15-19, 26-30 and 36-42, optionally with the exception of 1, 2, 3 or 4 mismatches.

In accordance with this example, the SSO may comprise a nucleotide sequence which is substantially complementary to SEQ ID NO: 15. For example, the SSO may comprise a nucleotide sequence which is complementary to SEQ ID NO: 15 with the exception of 4 base pair mismatches. For example, the SSO may comprise a nucleotide sequence which is complementary to SEQ ID NO: 15 with the exception of 3 base pair mismatches. For example, the SSO may comprise a nucleotide sequence which is complementary to SEQ ID NO: 15 with the exception of 2 base pair mismatches. For example, the SSO may comprise a nucleotide sequence which is complementary to SEQ ID NO: 15 with the exception of 1 base pair mismatch. In a particular example, the SSO may comprise a nucleotide sequence which is 100% complementary to SEQ ID NO: 15.

In accordance with this example, the SSO may comprise a nucleotide sequence which is substantially complementary to SEQ ID NO: 16. For example, the SSO may comprise a nucleotide sequence which is complementary to SEQ ID NO: 16 with the exception of 4 base pair mismatches. For example, the SSO may comprise a nucleotide sequence which is complementary to SEQ ID NO: 16 with the exception of 3 base pair mismatches. For example, the SSO may comprise a nucleotide sequence which is complementary to SEQ ID NO: 16 with the exception of 2 base pair mismatches. For example, the SSO may comprise a nucleotide sequence which is complementary to SEQ ID NO: 16 with the exception of 1 base pair mismatch. In a particular example, the SSO may comprise a nucleotide sequence which is 100% complementary to SEQ ID NO: 16.

In accordance with this example, the SSO may comprise a nucleotide sequence which is substantially complementary to SEQ ID NO: 17. For example, the SSO may comprise a nucleotide sequence which is complementary to SEQ ID NO: 17 with the exception of 4 base pair mismatches. For example, the SSO may comprise a nucleotide sequence which is complementary to SEQ ID NO: 17 with the exception of 3 base pair mismatches. For example, the SSO may comprise a nucleotide sequence which is complementary to SEQ ID NO: 17 with the exception of 2 base pair mismatches. For example, the SSO may comprise a nucleotide sequence which is complementary to SEQ ID NO: 17 with the exception of 1 base pair mismatch. In a particular example, the SSO may comprise a nucleotide sequence which is 100% complementary to SEQ ID NO: 17.

In accordance with this example, the SSO may comprise a nucleotide sequence which is substantially complementary to SEQ ID NO: 18. For example, the SSO may comprise a nucleotide sequence which is complementary to SEQ ID NO: 18 with the exception of 4 base pair mismatches. For example, the SSO may comprise a nucleotide sequence which is complementary to SEQ ID NO: 18 with the exception of 3 base pair mismatches. For example, the SSO may comprise a nucleotide sequence which is complementary to SEQ ID NO: 18 with the exception of 2 base pair mismatches. For example, the SSO may comprise a nucleotide sequence which is complementary to SEQ ID NO: 18 with the exception of 1 base pair mismatch. In a particular example, the SSO may comprise a nucleotide sequence which is 100% complementary to SEQ ID NO: 18.

In accordance with this example, the SSO may comprise a nucleotide sequence which is substantially complementary to SEQ ID NO: 19. For example, the SSO may comprise a nucleotide sequence which is complementary to SEQ ID NO: 19 with the exception of 4 base pair mismatches. For example, the SSO may comprise a nucleotide sequence which is complementary to SEQ ID NO: 19 with the exception of 3 base pair mismatches. For example, the SSO may comprise a nucleotide sequence which is complementary to SEQ ID NO: 19 with the exception of 2 base pair mismatches. For example, the SSO may comprise a nucleotide sequence which is complementary to SEQ ID NO: 19 with the exception of 1 base pair mismatch. In a particular example, the SSO may comprise a nucleotide sequence which is 100% complementary to SEQ ID NO: 19.

In accordance with this example, the SSO may comprise a nucleotide sequence which is substantially complementary to SEQ ID NO: 26. For example, the SSO may comprise a nucleotide sequence which is complementary to SEQ ID NO: 26 with the exception of 4 base pair mismatches. For example, the SSO may comprise a nucleotide sequence which is complementary to SEQ ID NO: 26 with the exception of 3 base pair mismatches. For example, the SSO may comprise a nucleotide sequence which is complementary to SEQ ID NO: 26 with the exception of 2 base pair mismatches. For example, the SSO may comprise a nucleotide sequence which is complementary to SEQ ID NO: 26 with the exception of 1 base pair mismatch. In a particular example, the SSO may comprise a nucleotide sequence which is 100% complementary to SEQ ID NO: 26.

In accordance with this example, the SSO may comprise a nucleotide sequence which is substantially complementary to SEQ ID NO: 27. For example, the SSO may comprise a nucleotide sequence which is complementary to SEQ ID NO: 27 with the exception of 4 base pair mismatches. For example, the SSO may comprise a nucleotide sequence which is complementary to SEQ ID NO: 27 with the exception of 3 base pair mismatches. For example, the SSO may comprise a nucleotide sequence which is complementary to SEQ ID NO: 27 with the exception of 2 base pair mismatches. For example, the SSO may comprise a nucleotide sequence which is complementary to SEQ ID NO: 27 with the exception of 1 base pair mismatch. In a particular example, the SSO may comprise a nucleotide sequence which is 100% complementary to SEQ ID NO: 27.

In accordance with this example, the SSO may comprise a nucleotide sequence which is substantially complementary to SEQ ID NO: 28. For example, the SSO may comprise a nucleotide sequence which is complementary to SEQ ID NO: 28 with the exception of 4 base pair mismatches. For example, the SSO may comprise a nucleotide sequence which is complementary to SEQ ID NO: 28 with the exception of 3 base pair mismatches. For example, the SSO may comprise a nucleotide sequence which is complementary to SEQ ID NO: 28 with the exception of 2 base pair mismatches. For example, the SSO may comprise a nucleotide sequence which is complementary to SEQ ID NO: 28 with the exception of 1 base pair mismatch. In a particular example, the SSO may comprise a nucleotide sequence which is 100% complementary to SEQ ID NO: 28.

In accordance with this example, the SSO may comprise a nucleotide sequence which is substantially complementary to SEQ ID NO: 29. For example, the SSO may comprise a nucleotide sequence which is complementary to SEQ ID NO: 29 with the exception of 4 base pair mismatches. For example, the SSO may comprise a nucleotide sequence which is complementary to SEQ ID NO: 29 with the exception of 3 base pair mismatches. For example, the SSO may comprise a nucleotide sequence which is complementary to SEQ ID NO: 29 with the exception of 2 base pair mismatches. For example, the SSO may comprise a nucleotide sequence which is complementary to SEQ ID NO: 29 with the exception of 1 base pair mismatch. In a particular example, the SSO may comprise a nucleotide sequence which is 100% complementary to SEQ ID NO: 29.

In accordance with this example, the SSO may comprise a nucleotide sequence which is substantially complementary to SEQ ID NO: 30. For example, the SSO may comprise a nucleotide sequence which is complementary to SEQ ID NO: 30 with the exception of 4 base pair mismatches. For example, the SSO may comprise a nucleotide sequence which is complementary to SEQ ID NO: 30 with the exception of 3 base pair mismatches. For example, the SSO may comprise a nucleotide sequence which is complementary to SEQ ID NO: 30 with the exception of 2 base pair mismatches. For example, the SSO may comprise a nucleotide sequence which is complementary to SEQ ID NO: 30 with the exception of 1 base pair mismatch. In a particular example, the SSO may comprise a nucleotide sequence which is 100% complementary to SEQ ID NO: 30.

In accordance with this example, the SSO may comprise a nucleotide sequence which is substantially complementary to SEQ ID NO: 36. For example, the SSO may comprise a nucleotide sequence which is complementary to SEQ ID NO: 36 with the exception of 4 base pair mismatches. For example, the SSO may comprise a nucleotide sequence which is complementary to SEQ ID NO: 36 with the exception of 3 base pair mismatches. For example, the SSO may comprise a nucleotide sequence which is complementary to SEQ ID NO: 36 with the exception of 2 base pair mismatches. For example, the SSO may comprise a nucleotide sequence which is complementary to SEQ ID NO: 36 with the exception of 1 base pair mismatch. In a particular example, the SSO may comprise a nucleotide sequence which is 100% complementary to SEQ ID NO: 36.

In accordance with this example, the SSO may comprise a nucleotide sequence which is substantially complementary to SEQ ID NO: 37. For example, the SSO may comprise a nucleotide sequence which is complementary to SEQ ID NO: 37 with the exception of 4 base pair mismatches. For example, the SSO may comprise a nucleotide sequence which is complementary to SEQ ID NO: 37 with the exception of 3 base pair mismatches. For example, the SSO may comprise a nucleotide sequence which is complementary to SEQ ID NO: 37 with the exception of 2 base pair mismatches. For example, the SSO may comprise a nucleotide sequence which is complementary to SEQ ID NO: 37 with the exception of 1 base pair mismatch. In a particular example, the SSO may comprise a nucleotide sequence which is 100% complementary to SEQ ID NO: 37.

In accordance with this example, the SSO may comprise a nucleotide sequence which is substantially complementary to SEQ ID NO: 38. For example, the SSO may comprise a nucleotide sequence which is complementary to SEQ ID NO: 38 with the exception of 4 base pair mismatches. For example, the SSO may comprise a nucleotide sequence which is complementary to SEQ ID NO: 38 with the exception of 3 base pair mismatches. For example, the SSO may comprise a nucleotide sequence which is complementary to SEQ ID NO: 38 with the exception of 2 base pair mismatches. For example, the SSO may comprise a nucleotide sequence which is complementary to SEQ ID NO: 38 with the exception of 1 base pair mismatch. In a particular example, the SSO may comprise a nucleotide sequence which is 100% complementary to SEQ ID NO: 38.

In accordance with this example, the SSO may comprise a nucleotide sequence which is substantially complementary to SEQ ID NO: 39. For example, the SSO may comprise a nucleotide sequence which is complementary to SEQ ID NO: 39 with the exception of 4 base pair mismatches. For example, the SSO may comprise a nucleotide sequence which is complementary to SEQ ID NO: 39 with the exception of 3 base pair mismatches. For example, the SSO may comprise a nucleotide sequence which is complementary to SEQ ID NO: 39 with the exception of 2 base pair mismatches. For example, the SSO may comprise a nucleotide sequence which is complementary to SEQ ID NO: 39 with the exception of 1 base pair mismatch. In a particular example, the SSO may comprise a nucleotide sequence which is 100% complementary to SEQ ID NO: 39.

In accordance with this example, the SSO may comprise a nucleotide sequence which is substantially complementary to SEQ ID NO: 40. For example, the SSO may comprise a nucleotide sequence which is complementary to SEQ ID NO: 40 with the exception of 4 base pair mismatches. For example, the SSO may comprise a nucleotide sequence which is complementary to SEQ ID NO: 40 with the exception of 3 base pair mismatches. For example, the SSO may comprise a nucleotide sequence which is complementary to SEQ ID NO: 40 with the exception of 2 base pair mismatches. For example, the SSO may comprise a nucleotide sequence which is complementary to SEQ ID NO: 40 with the exception of 1 base pair mismatch. In a particular example, the SSO may comprise a nucleotide sequence which is 100% complementary to SEQ ID NO: 40.

In accordance with this example, the SSO may comprise a nucleotide sequence which is substantially complementary to SEQ ID NO: 41. For example, the SSO may comprise a nucleotide sequence which is complementary to SEQ ID NO: 41 with the exception of 4 base pair mismatches. For example, the SSO may comprise a nucleotide sequence which is complementary to SEQ ID NO: 41 with the exception of 3 base pair mismatches. For example, the SSO may comprise a nucleotide sequence which is complementary to SEQ ID NO: 41 with the exception of 2 base pair mismatches. For example, the SSO may comprise a nucleotide sequence which is complementary to SEQ ID NO: 41 with the exception of 1 base pair mismatch. In a particular example, the SSO may comprise a nucleotide sequence which is 100% complementary to SEQ ID NO: 41.

In accordance with this example, the SSO may comprise a nucleotide sequence which is substantially complementary to SEQ ID NO: 42. For example, the SSO may comprise a nucleotide sequence which is complementary to SEQ ID NO: 42 with the exception of 4 base pair mismatches. For example, the SSO may comprise a nucleotide sequence which is complementary to SEQ ID NO: 42 with the exception of 3 base pair mismatches. For example, the SSO may comprise a nucleotide sequence which is complementary to SEQ ID NO: 42 with the exception of 2 base pair mismatches. For example, the SSO may comprise a nucleotide sequence which is complementary to SEQ ID NO: 42 with the exception of 1 base pair mismatch. In a particular example, the SSO may comprise a nucleotide sequence which is 100% complementary to SEQ ID NO: 42.

In one example, the SSO configured to induce skipping of exon 4 comprises a nucleotide sequence selected from the group of sequences set forth in SEQ ID NO: 49-53 and 59-65. In one particular example, the SSO configured to induce exon skipping of exon 4 comprises a sequence set forth in one of SEQ ID NOs: 60, 61 or 65.

For example, the SSO may comprise a nucleotide sequence which is substantially complementary to SEQ ID NO: 49. For example, the SSO may comprise a nucleotide sequence which is 85% complementary to SEQ ID NO: 49. For example, the SSO may comprise a nucleotide sequence which is 90% complementary to SEQ ID NO: 49. For example, the SSO may comprise a nucleotide sequence which is 95% complementary to SEQ ID NO: 49. In a particular example, the SSO may comprise a nucleotide sequence which is 100% complementary SEQ ID NO: 49.

For example, the SSO may comprise a nucleotide sequence which is substantially complementary to SEQ ID NO: 50. For example, the SSO may comprise a nucleotide sequence which is 85% complementary to SEQ ID NO: 50. For example, the SSO may comprise a nucleotide sequence which is 90% complementary to SEQ ID NO: 50. For example, the SSO may comprise a nucleotide sequence which is 95% complementary to SEQ ID NO: 50. In a particular example, the SSO may comprise a nucleotide sequence which is 100% complementary SEQ ID NO: 50.

For example, the SSO may comprise a nucleotide sequence which is substantially complementary to SEQ ID NO: 51. For example, the SSO may comprise a nucleotide sequence which is 85% complementary to SEQ ID NO: 51. For example, the SSO may comprise a nucleotide sequence which is 90% complementary to SEQ ID NO: 51. For example, the SSO may comprise a nucleotide sequence which is 95% complementary to SEQ ID NO: 51. In a particular example, the SSO may comprise a nucleotide sequence which is 100% complementary SEQ ID NO: 51.

For example, the SSO may comprise a nucleotide sequence which is substantially complementary to SEQ ID NO: 52. For example, the SSO may comprise a nucleotide sequence which is 85% complementary to SEQ ID NO: 52. For example, the SSO may comprise a nucleotide sequence which is 90% complementary to SEQ ID NO: 52. For example, the SSO may comprise a nucleotide sequence which is 95% complementary to SEQ ID NO: 52. In a particular example, the SSO may comprise a nucleotide sequence which is 100% complementary SEQ ID NO: 52.

For example, the SSO may comprise a nucleotide sequence which is substantially complementary to SEQ ID NO: 53. For example, the SSO may comprise a nucleotide sequence which is 85% complementary to SEQ ID NO: 53. For example, the SSO may comprise a nucleotide sequence which is 90% complementary to SEQ ID NO: 53. For example, the SSO may comprise a nucleotide sequence which is 95% complementary to SEQ ID NO: 53. In a particular example, the SSO may comprise a nucleotide sequence which is 100% complementary SEQ ID NO: 53.

For example, the SSO may comprise a nucleotide sequence which is substantially complementary to SEQ ID NO: 59. For example, the SSO may comprise a nucleotide sequence which is 85% complementary to SEQ ID NO: 59. For example, the SSO may comprise a nucleotide sequence which is 90% complementary to SEQ ID NO: 59. For example, the SSO may comprise a nucleotide sequence which is 95% complementary to SEQ ID NO: 59. In a particular example, the SSO may comprise a nucleotide sequence which is 100% complementary SEQ ID NO: 59.

For example, the SSO may comprise a nucleotide sequence which is substantially complementary to SEQ ID NO: 60. For example, the SSO may comprise a nucleotide sequence which is 85% complementary to SEQ ID NO: 60. For example, the SSO may comprise a nucleotide sequence which is 90% complementary to SEQ ID NO: 60. For example, the SSO may comprise a nucleotide sequence which is 95% complementary to SEQ ID NO: 60. In a particular example, the SSO may comprise a nucleotide sequence which is 100% complementary SEQ ID NO: 60.

For example, the SSO may comprise a nucleotide sequence which is substantially complementary to SEQ ID NO: 61. For example, the SSO may comprise a nucleotide sequence which is 85% complementary to SEQ ID NO: 61. For example, the SSO may comprise a nucleotide sequence which is 90% complementary to SEQ ID NO: 61. For example, the SSO may comprise a nucleotide sequence which is 95% complementary to SEQ ID NO: 61. In a particular example, the SSO may comprise a nucleotide sequence which is 100% complementary SEQ ID NO: 61.

For example, the SSO may comprise a nucleotide sequence which is substantially complementary to SEQ ID NO: 62. For example, the SSO may comprise a nucleotide sequence which is 85% complementary to SEQ ID NO: 62. For example, the SSO may comprise a nucleotide sequence which is 90% complementary to SEQ ID NO: 62. For example, the SSO may comprise a nucleotide sequence which is 95% complementary to SEQ ID NO: 62. In a particular example, the SSO may comprise a nucleotide sequence which is 100% complementary SEQ ID NO: 62.

For example, the SSO may comprise a nucleotide sequence which is substantially complementary to SEQ ID NO: 63. For example, the SSO may comprise a nucleotide sequence which is 85% complementary to SEQ ID NO: 63. For example, the SSO may comprise a nucleotide sequence which is 90% complementary to SEQ ID NO: 63. For example, the SSO may comprise a nucleotide sequence which is 95% complementary to SEQ ID NO: 63. In a particular example, the SSO may comprise a nucleotide sequence which is 100% complementary SEQ ID NO: 63.

For example, the SSO may comprise a nucleotide sequence which is substantially complementary to SEQ ID NO: 64. For example, the SSO may comprise a nucleotide sequence which is 85% complementary to SEQ ID NO: 64. For example, the SSO may comprise a nucleotide sequence which is 90% complementary to SEQ ID NO: 64. For example, the SSO may comprise a nucleotide sequence which is 95% complementary to SEQ ID NO: 64. In a particular example, the SSO may comprise a nucleotide sequence which is 100% complementary SEQ ID NO: 64.

For example, the SSO may comprise a nucleotide sequence which is substantially complementary to SEQ ID NO: 65. For example, the SSO may comprise a nucleotide sequence which is 85% complementary to SEQ ID NO: 65. For example, the SSO may comprise a nucleotide sequence which is 90% complementary to SEQ ID NO: 65. For example, the SSO may comprise a nucleotide sequence which is 95% complementary to SEQ ID NO: 65. In a particular example, the SSO may comprise a nucleotide sequence which is 100% complementary SEQ ID NO: 65.

Typically, an SSO of the disclosure will be synthesized in vitro. Methods of synthesizing oligonucleotides are known in the art. However, in some instances where modified nucleotides and backbones are not required, the SSOs of the disclosure may be expressed in vitro or in vivo in a suitable system, such as by a recombinant virus or cell.

### Modified Nucleotides

As described herein, an SSO of the disclosure may comprise one or more nucleotide or nucleobase (“base”) modifications or substitutions.

Exemplary modifications at the 2′ position of SSO's of the disclosure include: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. In one example, the SSO comprises one of the following at the 2′ position: O[(CH2)nO]mCH3, O(CH2)nOCH3, O(CH2)nNH2, O(CH2)nCH3, O(CH2)nONH2, and O(CH2)nON[(CH2)nCH3]2, where n and m are from 1 to about 10.

Further examples of modifications which may occur at the 2′ position of SSOs of the disclosure include: C1 to C10 lower alkyl, substituted lower alkyl, alkenyl, alkynyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties.

In one example, the SSO of the disclosure is modified to include 2′-methoxyethoxy (2′-O—CH2CH2OCH3 (also known as 2′-O-(2-methoxyethyl) or 2′-MOE) (Martin et al., 1995), that is, an alkoxyalkoxy group. In another example, the SSO of the disclosure is modified to include 2′-dimethylaminooxyethoxy, that is, a O(CH2)2ON(CH3)2 group (also known as 2′-DMAOE), or 2′-dimethylaminoethoxyethoxy (also known in the art as 2′-O-dimethyl-amino-ethoxy-ethyl or 2′-DMAEOE), that is, 2′-O—CH2-O—CH2-N(CH3)2.

Other modifications contemplated for SSOs of the disclosure include 2′-methoxy (2′-O—CH3), 2′-aminopropoxy (2′-OCH2CH2CH2NH2), 2′-allyl (2′-CH2-CH═CH2), 2′-O-allyl (2′-O—CH2-CH—CH2) and 2′-fluoro (2′-F). The 2′-modification may be in the arabino (up) position or ribo (down) position. In one example, a 2′-arabino modification is 2′-F.

Similar modifications may also be made at other positions on the SSO, particularly 3′ position of the sugar on 3′ terminal nucleotide or in 2′-5′ linked SSOs and 5′ position of the 5′ terminal nucleotide.

SSOs may also have sugar mimetics, such as cyclobutyl moieties in place of the pentofuranosyl sugar.

Representative United States patents that teach the preparation of such modified sugar structures include, but are not limited to, U.S. Pat. Nos. 4,981,957, 5,118,800, 5,319,080, 5,359,044, 5,393,878, 5,446,137, 5,466,786, 5,514,785, 5,519,134, 5,567,811, 5,576,427, 5,591,722, 5,597,909, 5,610,300, 5,627,053, 5,639,873, 5,646,265, 5,658,873, 5,670,633, 5,792,747, and 5,700,920.

A further modification of the sugar which is contemplated includes Locked Nucleic Acids (LNAs) in which the 2′-hydroxyl group is linked to 3′ or 4′ carbon atom of the sugar ring, thereby forming a bicyclic sugar moiety. In one example, the linkage is a methylene (—CH2-)n group bridging the 2′ oxygen atom and the 4′ carbon atom, wherein n is 1 or 2. LNAs and preparation thereof are described in WO 98/39352 and WO 99/14226.

Modified nucleobases include other synthetic and natural nucleobases such as, for example, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—CC—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Further modified nucleobases include tricyclic pyrimidines, such as phenoxazine cytidine (1H-pyrimido[5,4-b][1,4]benzoxazin-2 (3H)-one), phenothiazine cytidine (1H-pyrimido[5,4-b][1,4]benzothiazin-2 (3H)-one), G-clamps such as, for example, a substituted phenoxazine cytidine (e.g., 9-(2-aminoethoxy)-H-pyrimido[5,4-b][1,4]benzoxazin-2 (3H)-one), carbazole cytidine (2H-pyrimido[4,5-b]indol-2-one), pyridoindole cytidine (H-pyrido[3′,2′:4,5]pyrrolo[2,3-d]pyrimidin-2-one).

Modified nucleobases may also include those in which the purine or pyrimidine base is replaced with other heterocycles, for example, 7-deaza-adenine, 7-deazaguanosine, 2-aminopyridine and 2-pyridone. Further nucleobases include those disclosed in U.S. Pat. No. 3,687,808, those disclosed in J. I. Kroschwitz (editor), The Concise Encyclopedia of Polymer Science and Engineering, pages 858-859, John Wiley and Sons (1990), those disclosed by Englisch et al. (1991), and those disclosed by Y. S. Sanghvi, Chapter 15: Antisense Research and Applications, pages 289-302, S. T. Crooke, B. Lebleu (editors), CRC Press, 1993.

Certain nucleobases are particularly useful for increasing the binding affinity of the oligonucleotide. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. Such nucleobases are contemplated herein. In one example, these nucleobase substitutions are combined with 2′-O-methoxyethyl sugar modifications.

Representative United States patents that teach the preparation the above noted modified nucleobases as well as other modified nucleobases include, but are not limited to, U.S. Pat. Nos. 3,687,808, 4,845,205, 5,130,302, 5,134,066, 5,175,273, 5,367,066, 5,432,272, 5,457,187, 5,459,255, 5,484,908, 5,502,177, 5,525,711, 5,552,540, 5,587,469, 5,594,121, 5,596,091, 5,614,617, 5,645,985, 5,830,653, 5,763,588, 6,005,096, 5,681,941 and 5,750,692.

Unless stated to the contrary, reference to an A, T, G, U or C can either mean a naturally occurring base or a modified version thereof.

### Backbones

SSOs of the present disclosure may also include those having modified backbones or non-natural inter-nucleoside linkages. SSOs having modified backbones include those that retain a phosphorus atom in the backbone and those that do not have a phosphorus atom in the backbone.

Modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Oligonucleotides having inverted polarity comprise a single 3′ to 3′ linkage at 3′-most internucleotide linkage, that is, a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808, 4,469,863, 4,476,301, 5,023,243, 5,177,196, 5,188,897, 5,264,423, 5,276,019, 5,278,302, 5,286,717, 5,321,131, 5,399,676, 5,405,939, 5,453,496, 5,455,233, 5,466,677, 5,476,925, 5,519,126, 5,536,821, 5,541,306, 5,550,111, 5,563,253, 5,571,799, 5,587,361, 5,194,599, 5,565,555, 5,527,899, 5,721,218, 5,672,697 and 5,625,050.

Modified oligonucleotide backbones that do not include a phosphorus atom therein include, for example, backbones formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH2 component parts.

Representative United States patents that teach the preparation of the above oligonucleotides include, but are not limited to, U.S. Pat. Nos. 5,034,506, 5,166,315, 5,185,444, 5,214,134, 5,216,141, 5,235,033, 5,264,562, 5,264,564, 5,405,938, 5,434,257, 5,466,677, 5,470,967, 5,489,677, 5,541,307, 5,561,225, 5,596,086, 5,602,240, 5,610,289, 5,602,240, 5,608,046, 5,610,289, 5,618,704, 5,623,070, 5,663,312, 5,633,360, 5,677,437, 5,792,608, 5,646,269 and 5,677,439.

### Testing of Candidate SSOs

The SSOs of the present disclosure can be conveniently and routinely made through the well-known technique of solid phase synthesis. Any other means for such synthesis known in the art may additionally or alternatively be employed. It is well known to use similar techniques to prepare SSOs such as the phosphorothioates and alkylated derivatives. In this regard, the present disclosure is not limited by the method of SSO synthesis.

Methods of SSO purification and analysis will also be known to those skilled in the art. Analysis methods include capillary electrophoresis (CE) and electrospray-mass spectroscopy. Such synthesis and analysis methods can be performed in multi-well plates. The method of the disclosure is not limited by the method of SSO purification.

Once synthesized, candidate SSOs can be tested for their desired activity using standard procedures and techniques known in the art. For example, screening of candidates may involve administering the candidate SSOs to cells expressing midkine in vitro (e.g., via transfection) and determining the effect of the candidate SSO on expression of midkine in those cells at the mRNA and/or protein level. Detection and quantification of full length and truncated forms of midkine protein can then be performed using standard molecular techniques, such as protein gel electrophoresis and Western blotting. Similarly, detection and quantification of full length and truncated forms of midkine mRNA can then be undertaken using mRNA reverse transcription PCR (RT-PCR) followed by agarose gel electrophoresis, as known in the art. In another example, candidate SSOs may be administered to an animal (e.g., an animal model of midkine-related disease or condition which is known to express midkine), and the animal can be screened for the amount and species midkine mRNA and/or midkine protein expressed. Functional assay may also be performed to assess the effect of candidate SSOs on midkine function in animals to which they are administered. In another example, a candidate SSO may simply be tested for its ability to hybridize to a target polynucleotide (such as mRNA).

### Compositions

The present disclosure also provides a composition comprising one or more SSOs as described herein for administration. A composition of the disclosure may comprise one of the SSOs described herein, or may comprise a plurality of the SSO described herein (e.g., as a cocktail). Accordingly, a plurality of SSOs of the disclosure may be provided in a single composition. Alternatively, a plurality of SSOs of the disclosure may be provided in multiple compositions. For example, each of the SSOs of the plurality may be provided in separate compositions. Alternatively, at least one SSO of the plurality may be provided separately and two or more of the plurality provided together in a composition. A plurality of SSOs in accordance with the present disclosure may comprise up to ten SSOs, such as two SSOs or three SSOs or four SSOs or five SSOs or six SSOs or seven SSOs or eight SSOs or nine SSOs or ten SSOs.

In one example, the pharmaceutical composition comprises a plurality of SSOs which each induce skipping of exon 3 of midkine. In another example, the pharmaceutical composition comprises a plurality of SSOs which each induce skipping of exon 4 of midkine. In yet another example, the pharmaceutical composition comprises at least one SSO which induces skipping of exon 3 of midkine and at least one SSO which induces skipping of exon 4 of midkine. Exemplary SSOs for skipping exon 3 and exon 4 of midkine respectively are described herein and shall be taken to apply mutatis mutandis to examples describing pharmaceutical compositions of the disclosure.

SSOs of the disclosure may be admixed, encapsulated, conjugated (such as fused) or otherwise associated with other molecules, molecule structures or mixtures of compounds, resulting in, for example, liposomes, Lipid nanoparticles (LNPs), receptor-targeted molecules, oral, rectal, topical, inhalable, injectable or other formulations, for assisting in uptake, distribution and/or absorption [Paunovska K et al (2022) Nature Reviews Genetics 4:1-16]. Representative United States patents that teach the preparation of such uptake, distribution and/or absorption-assisting formulations include, but are not limited to, U.S. Pat. Nos. 5,108,921, 5,354,844, 5,416,016, 5,459,127, 5,521,291, 5,543,158, 5,547,932, 5,583,020, 5,591,721, 4,426,330, 4,534,899, 5,013,556, 5,108,921, 5,213,804, 5,227,170, 5,264,221, 5,356,633, 5,395,619, 5,416,016, 5,417,978, 5,462,854, 5,469,854, 5,512,295, 5,527,528, 5,534,259, 5,543,152, 5,556,948, 5,580,575, and 5,595,756.

As used herein, the term “lipid nanoparticle” or “LNP” shall be understood to refer to lipid-based particles having at least one dimension on the order of nanometers (e.g., 1-1,000 nm). LNPs may comprise an ionizable cationic compound, a neutral lipid, charged lipid, sterol and PEGylated lipid. In some examples, the lipid nanoparticle or LNP may be selected from liposomes or vesicles, where an aqueous volume is encapsulated by amphipathic lipid bilayers (e.g., single; unilamellar or 10 multiple; multilamellar), micelle-like lipid nanoparticles having a non-aqueous core and solid lipid nanoparticles. In some examples, the lipid nanoparticle or LNP may have a structure that includes a single monolayer or bilayer of lipids that encapsulates a solid phase. In other examples, the lipid nanoparticle or LNP does not have an aqueous phase or other liquid phase in its interior.

LNPs may comprise bilayer stabilizing component (BSC) such as an ATTA-lipid or a PEG-lipid, such as PEG coupled to dialkyloxypropyls (PEG-DAA) as described in, e.g., WO 05/026372, PEG coupled to diacylglycerol (PEG-DAG) as described in, e.g., U.S. Patent Publication Nos. 20030077829 and 2005008689, PEG coupled to dimyristoylglecerol (PEG-DMG) as described in, e.g., Abrams et. al., Molecular Therapy 2010, 18(1), 171, PEG coupled to phosphatidylethanolamine (PE) (PEG-PE), or PEG conjugated to 1,2-Di-O-hexadecyl-sn-glyceride (PEG-DSG), or a mixture thereof (see, U.S. Pat. No. 5,885,613). For example, the BSC may be a conjugated lipid that inhibits aggregation of the lipid nanoparticle.

For example, the LNP may comprise a neutral lipid, e.g., a phospholipid or an analog or derivative thereof, a structural lipid, e.g., selected from the group consisting of cholesterol, fecosterol, sitosterol, ergosterol, campesterol, stigmasterol, brassicasterol, tomatidine, ursolic acid, alpha-tocopherol, and mixtures thereof, or a PEG lipid, e.g., selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

SSOs of the disclosure may be conjugated to one or more moieties or groups which enhance the activity, cellular distribution or cellular uptake of the oligonucleotide. These moieties or groups may be covalently bound to functional groups such as primary or secondary hydroxyl groups. Exemplary moieties or groups include intercalators, reporter molecules, polyamines, polyamides, polyethylene glycols, polyethers, peptides, N-acetylgalactosamine, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins and dyes. SSOs of the disclosure may formulated with one or more pharmaceutically acceptable carriers, diluents or excipients to assist with administration. Alternatively, the SSOs of the disclosure can be lyophilized for storage and reconstituted in a suitable carrier prior to use according to art-known lyophilization and reconstitution techniques.

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, and/or other problem or complication, commensurate with a reasonable benefit/risk ratio.

In general terms, by “carrier, diluent or excipient” is meant a solid or liquid filler, binder, diluent, encapsulating substance, emulsifier, wetting agent, solvent, suspending agent, coating or lubricant that may be safely administered to any mammal, e.g., a human. Depending upon the particular route of administration, a variety of acceptable carriers, diluents or excipients, known in the art may be used, as for example described in Remington's Pharmaceutical Sciences (Mack Publishing Co. N.J. USA, 1991). The pharmaceutically acceptable carrier may be solid or liquid. Useful examples of pharmaceutically acceptable carriers include, but are not limited to, diluents, solvents, surfactants, excipients, suspending agents, buffering agents, lubricating agents, adjuvants, vehicles, emulsifiers, absorbants, dispersion media, coatings, stabilizers, protective colloids, adhesives, thickeners, thixotropic agents, penetration agents, sequestering agents, isotonic and absorption delaying agents that do not affect the activity of the active agents of the disclosure.

In one example, the pharmaceutical carrier is water for injection (WFI) and the pharmaceutical composition is adjusted to pH 7.4, 7.2-7.6. In one example, the salt is a sodium or potassium salt.

The SSOs may contain chiral (asymmetric) centres or the molecule as a whole may be chiral. The individual stereoisomers (enantiomers and diastereoisomers) and mixtures of these are within the scope of the present disclosure.

SSOs of the disclosure may be formulated as pharmaceutically acceptable salts, esters, or salts of the esters, or any other compounds which, upon administration are capable of providing (directly or indirectly) the biologically active metabolite. The term “pharmaceutically acceptable salts” as used herein refers to physiologically and pharmaceutically acceptable salts of the SSO that retain the desired biological activities of the parent compounds and do not impart undesired toxicological effects upon administration. Examples of pharmaceutically acceptable salts and their uses are further described in U.S. Pat. No. 6,287,860.

In one example, an SSO of the disclosure can be complexed with a complexing agent to increase its cellular uptake. An example of a complexing agent includes cationic lipids. Cationic lipids can be used to deliver SSOs to cells.

The term “cationic lipid” includes lipids and synthetic lipids having both polar and nonpolar domains and which are capable of being positively charged at or around physiological pH and which bind to polyanions, such as nucleic acids, and facilitate the delivery of nucleic acids into cells. In general cationic lipids include saturated and unsaturated alkyl and alicyclic ethers and esters of amines, amides, or derivatives thereof. Straight-chain and branched alkyl and alkenyl groups of cationic lipids can contain, e.g., from 1 to about 25 carbon atoms. Preferred straight chain or branched alkyl or alkene groups have six or more carbon atoms. Alicyclic groups include cholesterol and other steroid groups. Cationic lipids can be prepared with a variety of counterions (anions) including, e.g., Cl—, Br—, I—, F—, acetate, trifluoroacetate, sulfate, nitrite, and nitrate.

Examples of cationic lipids include polyethylenimine, polyamidoamine (PAMAM) starburst dendrimers, Lipofectin (a combination of DOTMA and DOPE), Lipofectase, LIPOFECTAMINE™ (e.g., LIPOFECTAMINE™ 3000), DOPE, Cytofectin (Gilead Sciences, Foster City, Calif.), and Eufectins (JBL, San Luis Obispo, Calif.). Exemplary cationic liposomes can be made from N-[1-(2,3-dioleoloxy)-propyl]-N,N,N-trimethylammonium chloride (DOTMA), N-[1-(2,3-dioleoloxy)-propyl]-N,N,N-trimethylammonium methylsulfate (DOTAP), 3.beta.-[N—(N′,N′-dimethylaminoethane) carbamoyl]cholesterol (DC-Chol), 2,3,-dioleyloxy-N-[2 (sperminecarboxamido)ethyl]-N,N-dimethyl-1-propanaminium trifluoroacetate (DOSPA), 1,2-dimyristyloxypropyl-3-dimethyl-hydroxyethyl ammonium bromide; and dimethyldioctadecylammonium bromide (DDAB). Oligonucleotides can also be complexed with, e.g., poly (L-lysine) or avidin and lipids may, or may not, be included in this mixture, e.g., steryl-poly (L-lysine).

Cationic lipids have been used in the art to deliver oligonucleotides to cells (see, e.g., U.S. Pat. Nos. 5,855,910; 5,851,548; 5,830,430; 5,780,053; 5,767,099; Lewis et al., 1996; Hope et al., 1998). Other lipid compositions which can be used to facilitate uptake of the instant SSOs can be used in connection with the methods of the disclosure. In addition to those listed above, other lipid compositions are also known in the art and include, e.g., those taught in U.S. Pat. Nos. 4,235,871; 4,501,728; 4,837,028; 4,737,323.

In one example, lipid compositions can further comprise agents, e.g., viral proteins to enhance lipid-mediated transfections of oligonucleotides. In another example, N-substituted glycine oligonucleotides (peptoids) can be used to optimize uptake of oligonucleotides.

In another example, a composition for delivering SSOs of the disclosure may comprise a peptide having from between about one to about four basic residues. These basic residues can be located, e.g., on the amino terminal, C-terminal, or internal region of the peptide. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine (can also be considered non-polar), asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Apart from the basic amino acids, a majority or all of the other residues of the peptide can be selected from the non-basic amino acids, e.g., amino acids other than lysine, arginine, or histidine. In particular examples, a preponderance of neutral amino acids with long neutral side chains are used.

In one example, SSOs of the disclosure are modified by attaching a peptide sequence that assists with transport of the oligonucleotide into a cell, referred to herein as a “transporting peptide” or “cell penetrating peptide (CPP)”. In one example, an SSO of the disclosure is covalently attached to a transporting peptide or a CPP.

### Dosages and Regimens

For the prevention or treatment of a disease or condition or relapse thereof, the appropriate dosage of an active agent (e.g., an SSO of the disclosure), will depend on the type of disease to be treated, the severity and course of the disease, whether the active agent is administered for preventive or therapeutic purposes, previous therapy, the patient's clinical history and response to the active agent, and the discretion of the attending physician. Typically, a therapeutically effective amount of the SSO will be administered. The particular dosage regimen, i.e., dose, timing, and repetition, will depend on the particular individual and that individual's medical history as assessed by a physician. Typically, a clinician will administer an active agent (e.g., antibody or conjugate comprising same) until a dosage is reached that achieves the desired result.

As used herein, the terms “treating”, “treat” or “treatment” include administering a therapeutically effective amount of a compound(s) described herein sufficient to reduce or eliminate at least one symptom of a disease, disorder or condition.

As used herein, the terms “preventing”, “prevent” or “prevention” include administering a therapeutically effective amount of a compound(s) described herein sufficient to stop or hinder the development of at least one symptom of a disease, disorder or condition.

The terms “therapeutically effective amount” and “effective amount” describe a quantity of a specified agent, such as an oligonucleotide of the disclosure, sufficient to achieve a desired effect in a subject or cell being treated or contacted with that agent. For example, this can be the amount of a composition comprising one or more agents that inhibit the activity of one or more nucleic acid sensors described herein, necessary to reduce, alleviate and/or prevent a disease, disorder or condition. In some examples, a “therapeutically effective amount” is sufficient to reduce or eliminate a symptom of a disease, disorder or condition. In another example, a “therapeutically effective amount” or “effective amount” is an amount sufficient to achieve a desired biological effect, for example, an amount that is effective to decrease or prevent a senescence-associated disease, disorder or condition or inhibit or prevent senescence in a cell.

Ideally, a therapeutically effective amount of an agent is an amount sufficient to induce the desired result without causing a substantial cytotoxic effect in the subject. The effective amount of an agent useful for reducing, alleviating and/or preventing a disease, disorder or condition will be dependent on the subject being treated, the type and severity of any associated symptoms and the manner of administration of the therapeutic composition.

Generally, the dosage will vary with the age, condition, sex and extent of the disease in the patient and can be determined by one of skill in the art. The dosage can be adjusted by the individual physician in the event of any complication. For in vivo administration of the SSOs described herein, normal dosage amounts may vary from about 10 ng/kg up to about 100 mg/kg of an individual's body weight or more per day. Exemplary dosages and ranges thereof are described herein. For repeated administrations over several days or longer, depending on the severity of the disease or disorder to be treated, the treatment can be sustained until a desired suppression of symptoms is achieved.

Dosages for a particular SSO may be determined empirically in mammals who have been given one or more administrations of the respective SSO. To assess efficacy of an SSO of the disclosure, a clinical symptom of a disease or condition being treated e.g., cancer, can be monitored. For example, efficacy of an SSO of the disclosure in treatment of cancer may be assessed based on tumour size and/or using diagnostic, prognostic or predictive biomarkers of cancer.

Administration of an SSO according to the methods of the present disclosure can be continuous or intermittent, depending, for example, on the recipient's physiological condition, whether the purpose of the administration is therapeutic or prophylactic, and other factors known to skilled practitioners. The administration of an SSO may be essentially continuous over a preselected period of time or may be in a series of spaced doses.

A variety of routes of administration are possible including, but not necessarily limited to, oral, dietary, topical, parenteral (e.g., intravenous, intraarterial, intramuscular, subcutaneous injection), inhalation (e.g., intrabronchial, intraocular, intranasal or oral inhalation, intranasal drops), depending on the disease or condition to be treated. Other suitable methods of administration can also include rechargeable or biodegradable devices and slow-release biologic or synthetic polymeric devices.

### Uses

SSOs of the present disclosure have the ability to inhibit midkine function and can therefore be used as therapeutic and preventative drugs for midkine-related diseases and disorders.

In one example, the present disclosure provides a method for inhibiting an interaction between human midkine and a ligand thereof on the surface of or in a cell, said method comprising exposing the cell to one or more SSOs described herein or to the pharmaceutical composition described herein. It will be appreciated by the skilled person that the method may be performed in vitro, ex vivo or in vivo.

The cell may be any known in the art that expresses midkine. By way of example, the cell can be an immune cell, such as T cells, B cells, natural killer cells, neutrophils, eosinophils, mast cells, basophils, monocytes, macrophages and dendritic cells; endothelial cells; or neurones. In oncology the cells can be malignant tumour cells or stromal cells, such as fibroblasts or endothelial cells.

In another example, the disclosure provides a method for inhibiting human midkine activity in a cell, said method comprising exposing the cell to one or more SSOs described herein or to the pharmaceutical composition described herein.

As used herein, the term “inhibit” shall be taken to mean hinder, reduce, restrain or prevent midkine activity in a cell relative to midkine activity in a cell in which the SSO of the present disclosure is absent.

Midkine activity may be inhibited in any measurable amount. Inhibition of midkine activity may be complete or may be partial. Thus, the methods disclosed herein may comprise at least partial inhibition of midkine activity. For example, the activity of midkine may be reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100% following contacting the cell with an effective amount of the SSO (e.g., relative to the same measurement of activity before contact with the SSO).

In another example, the disclosure provides a method for treating or preventing a midkine-related disease or disorder in a subject in need thereof, said method comprising administering to the subject one or more SSOs described herein or the pharmaceutical composition described herein.

In yet another example, the disclosure provides for use of one or more SSOs described herein, or the pharmaceutical composition described herein, in the preparation of a medicament for treatment or prevention of a midkine-related disease or disorder,

The term “midkine-related disease” refers to a disease involving midkine functions. Examples of such diseases include: diseases attributed to cell growth or angiogenesis, such as cancers (esophageal cancer, thyroid cancer, urinary bladder cancer, colon cancer, stomach cancer, pancreatic cancer, thoracic cancer, liver cancer, lung cancer, breast cancer, neuroblastoma, glioblastoma, uterine cancer, ovarian cancer, prostatic cancer, and Wilms tumor) and endometriosis; inflammatory diseases or diseases attributed to recruitment of inflammatory cells, suppression of regulatory T cell function, activation of neutrophils, dysfunctional orientation of macrophages or T cells such as inflammatory diseases of the kidney, acute renal failure, chronic kidney diseases, osteoporosis, sepsis, arthritis, autoimmune disease (organ-specific autoimmune disease, etc.), rheumatic arthritis (rheumatoid arthritis (RA) or osteoarthritis (OA)), multiple sclerosis (relapsing-remitting multiple sclerosis, etc.), inflammatory bowel disease (Crohn disease, etc.), systemic lupus erythematosus (SLE), progressive systematic sclerosis (PSS), Sjogren's syndrome, polymyositis (PM), dermatomyositis (DM), polyarteritis nodosa (PN), thyroid disease (Graves disease, etc.), Guillain-Barre syndrome, primary biliary cirrhosis (PBC), idiopathic thrombocytopenia purpura, autoimmune hemolytic anemia, experimental autoimmune myasthenia gravis (EAMG), amyotrophic lateral sclerosis (ALS), type I diabetes mellitus, transplant rejection, postoperative adhesion, endometriosis, psoriasis, lupus, allergy, asthma, acute respiratory distress syndrome; ventilation induced lung injury; and occlusive vascular diseases or diseases attributed to vascular intimal thickening, such as post-revascularization restenosis, coronary occlusive disease, cerebrovascular occlusive disease, renovascular occlusive disease, peripheral occlusive disease, arteriosclerosis, and cerebral infarction. In one example, the midkine-related disease or disorder is selected from an autoimmune disease, cancer, or an inflammatory disease.

In particular examples, the disease to be treated or prevented using an SSO of the disclosure demonstrates increased, excessive or abnormal MDK expression, accumulation, activity and/or signalling. Such diseases are described herein, albeit without limitation thereto. In another example, the SSOs of the disclosure may be used in methods of preventing or inhibiting inflammation associated with administration of a therapeutic SSO, such as those known in the art, to a subject. In particular, the SSOs described herein may be used in the prevention or inhibition of inflammation mediated by one or more nucleic acid sensors (e.g., TLR3, TLR7, TLR8, TLR9, cGAS, RIG-I) during or following administration of the therapeutic SSO. It is envisaged that the inflammation may involve or include any cells, tissues or organs of the body. In particular examples, the inflammation is or comprises hepatic inflammation. To this end, the therapeutic SSO may be conjugated to N-acetylgalactosamine (GalNAc), which enhances asialoglycoprotein receptor (ASGR)-mediated uptake into liver hepatocytes (Nair et al., 2014), and thereby enabling their specific targeting to the liver.

In particular examples, therapeutically effective amounts of the therapeutic SSO and the SSO of the disclosure may be administered simultaneously, concurrently, sequentially, successively, alternately or separately in any particular combination and/or order.

In accordance with the above-mentioned uses, the SSOs of the disclosure may be administered to an animal. For example, the animal (or subject) to which the SSO is administered may be a mammal, avian, chordate, amphibian or reptile. In one example, the animal is a mammal. Exemplary mammalian subjects include but are not limited to human, primate, livestock (e.g. sheep, cow, chicken, horse, donkey, pig), companion animals (e.g. dogs, cats), laboratory test animals (e.g. mice, rabbits, rats, guinea pigs, hamsters), captive wild animal (e.g. fox, deer). In one example, the mammal is a human.

### Combination Therapies

The SSOs and compositions of the present disclosure can also be administered as part of a combinatorial therapy with other agents useful for treating a disease or condition, e.g., cancer, either as combined or additional treatment steps or as additional components of a therapeutic formulation. Such other therapies/agents will be well-known to those skilled in the art.

For example, the other compound is an anti-inflammatory or immunomodulatory drug. Alternatively, or additionally, the other compound is an immunosuppressant. Alternatively, or additionally, the other compound is a chemotherapeutic agent, such as carboplatin.

So that preferred examples of the present disclosure may be fully understood and put into practical effect, reference is made to the following non-limiting examples.

TABLE 1
Midkine primer sets

| Region ID | Sequence (5′-3′) | SEQ ID NO: |
|---|---|---|
| hMDK Ex1F | AGACCGGTTCTGGAGACAAA | SEQ ID NO: 1 |
| hMDK Ex5R | GCTCTGGGACTCACATTGCT | SEQ ID NO: 2 |
| hMDK Ex5R | TCCCAGAATCCCTTGTCCCT | SEQ ID NO: 3 |
| hMDK Ex2F | AGGCTTCCTCCTCCTCACC | SEQ ID NO: 4 |
| hMDK Ex2F | CGCGGTCGCCAAAAAGAAAG | SEQ ID NO: 5 |

TABLE 2
Target regions within MDK protein

| Region | | SEQ ID |
|---|---|---|
| ID | Region sequence (5′-3′) | NO: |
| MDK | CGGCTTCCCGCTTCCCGCGGAGAACAACAATGAAAGTGAAAGAGGGGTG | 6 |
| full | GGGCGGGGGCGAGCCCGGGTTCTGTGGCCCATTTGCCCTGTGGCCTTGAG | |
| length | CAAGCCCCTCCCCCAGGCCTCGGGGGCTCTCCCGGTTTGGGGGAACCGGG | |
| | CGAGGCAATGCCACAGGCCCAGGGTTAGAGGGGGTGGGCACTTGCAGCT | |
| | GCCGATGTGGCTGGATCTGGAACTTCTCAGACGGCTCCTGTCAGCGCCAA | |
| | GTTTCACCAAATCCAGGCCTGCGGGCTCCTCCCCCAGGACCCCCACTCGC | |
| | AGTCCCTCAAGCCTGTGCTCCCGGAAAGGCACTGGGCGACCGCACCCGTG | |
| | GCTTTCTCTGGGCGACCGGGTCCCAGACTCCCCCCAGCACAGCAGAGCGC | |
| | TTCCCTGCCCACCCGCGGAAACCGCCCCAGGTGGGCCGCGCCCCCTCCCC | |
| | AGCAGCCAGCAGGGCGCCAGGGCTGAGCCGGCCGTGGAGGGGAGCGGGT | |
| | CCCGCGGGTTATACAGGCGCCGGGGCTCCGCGGCAGGCAAGAGAAGCTG | |
| | AGGCCTGAGAACGGCCCGGGCCTTGGCGTACGGCAGGGGACGACCTGGG | |
| | ATGGGGGCAGCGGGCGGCGGCGCAGGGAGTGGGCCGGGGGCCGGTGTGC | |
| | GCGGGCGGGACGGGGCCCGGGGTCGGGAGACCACCGCTCGGAAGATGGG | |
| | GCCGGGAGAGGCCGCCGTCGCAGCGCAGAGGGCACCGGGGGGAGACG | |
| | CGAGGACGCGGGGCCGGGAACACGGACGCCGGAGTAGAAGCGCGGGGG | |
| | GCGCGGGCTGGAGCGGGGGCGGGGACGCCGGGGTCGGGGGCGGTGCGG | |
| | GTTTGAGGGGAGGGGGCGGGGCGGGTCCTTCCCTGGGGGGGTGGGGAGA | |
| | GGGGGCGGGGGCCCATGTGACCGGCTCAGACCGGTTCTGGAGACAAAAG | |
| | GGGCCGCGGCGGCCGGAGCGGGACGGGCCCGGCGCGGGAGGGAGCGAA | |
| | GCAGCGCGGGCAGCGAGCGAGTGAGCGCGCGGCGGCCCCTGGTCCGCCC | |
| | GGCCGCGGCCGATCTAGGGGCTGGGGGCTGGAGGCGGGGGTGGGGGTCT | |
| | GAGCTGCGTCCTGGGCTCGAGGCGTCCCCCGGGGAGTCGCCTCTTAGCGG | |
| | TGCGTCCGGGCTAGCGGCGAGGGGCCGCCCCAAGTCTTCCCACCGCCGCC | |
| | ACCTTAGCAGCCCGACTTGGGGCCTGGAAAGTGGAGCACGCGGAGGTGG | |
| | GAGGGCCCTGCACGCGGCCCCCGGTGGGGAAGGGGACGGGCCAGGGATT | |
| | CAGACTCGGGCTCTCCCCTCAGGATGCAGCACCGAGGCTTCCTCCTCCTC | |
| | ACCCTCCTCGCCCTGCTGGCGCTCACCTCCGCGGTCGCCAAAAAGAAAGG | |
| | TGATGGGGGATGATCGAAGGAGGGCTGGGGACGGGCAGGCGAGGCCCCT | |
| | CCACTTCTGGGCTGGGCCGCCTGGGTTCCTAGCCTGGAACCCCAGGAAGG | |
| | CGGCTCCCGAGGGAGTCTCCCCGTGCCCCAGTCCTGAACTCTGTTCCTCG | |
| | CGCGTTGTAGATAAGGTGAAGAAGGGCGGCCCGGGGAGCGAGTGCGCTG | |
| | AGTGGGCCTGGGGGCCCTGCACCCCCAGCAGCAAGGATTGCGGCGTGGG | |
| | TTTCCGCGAGGGCACCTGCGGGGCCCAGACCCAGCGCATCCGGTGCAGG | |
| | GTGCCCTGCAACTGGAAGAAGGAGTTTGGAGGTGAGGCGGGGCGCAGTC | |
| | AGAGGGCAGGAGACGGGGGGCACAGCCTCGCCGAAGCCTGGGCGGACCC | |
| | TTGGCGGAGGGCGGGGCCGCGGGCCGCGCAGCGCTGACCTGGGCCGCTC | |
| | TCTCGCCAGCCGACTGCAAGTACAAGTTTGAGAACTGGGGTGCGTGTGAT | |
| | GGGGGCACAGGCACCAAAGTCCGCCAAGGCACCCTGAAGAAGGCGCGCT | |
| | ACAATGCTCAGTGCCAGGAGACCATCCGCGTCACCAAGCCCTGCACCCCC | |
| | AAGACCAAAGCAAAGGCCAAAGGTCAGCGAAAGGAGAAGGGGGTGGGG | |
| | CTGTCGCGGGGGGCTGCCCCCCCCCCCCCCCGCCTGTGAGGGGACAATTC | |
| | CAAGTTAAACCTTAAGTTTTGAGTCCTGGCCAGTGGCTTCCTGACATCGC | |
| | CTCACTTGGCTTCCCTGCCTGGAAAAGTCTGAAGATGGGCACTACAAGAG | |
| | AGGCCGCAGGTGATGCTGGGGACATAAATCCTCCCTGGCCCAAATAGGG | |
| | ACCAACTCAAACTACTCCATTGGAGCATCTGGCTTAGGACCCAGGGAGAG | |
| | TGTCCTGGAACGGCTTGCCTTTGTCAGCTCTCCAGCCACGGGCAGCATTT | |
| | GGTCAGCTCTGCCCTTTCTAGTGTTGGGAGGAGGTCAAGGCCCACCCTGG | |
| | GCCTCTCAGCTCACTCGTGACTCAGCCCAGCGAGGCCAGCAGGGCAGGG | |
| | GTGAATCTGCCCGCTTCTCAGGTGAGGAGGCTGAGGATGCCCAGGGCTGC | |
| | TGTGACCAGGACTAGGACTGGAAACTTGAAGGTTTTCTGATCCCAAGTGG | |
| | AAATAGGAAGCTGGGGATGTCCCATGTCCACATCACAATGGCTGCCCCAT | |
| | CCCCTGCTTCCGAGTCAGCTGATTGGAAACCACTAGGGGGCAGAATCTTC | |
| | TCCTTCCCTGATGCCCGGGTGTTTGTGGAGCCGGCGGTCTGCAATGGGTC | |
| | AGCCTAACTGCTGATATGGTATTAATATTTCTTTCTTGTTTTACAGCCAAG | |
| | AAAGGGAAGGGAAAGGACTAGACGCCAAGCCTGGATGCCAAGGAGCCC | |
| | CTGGTGTCACATGGGGCCTGGCCCACGCCCTCCCTCTCCCAGGCCCGAGA | |
| | TGTGACCCACCAGTGCCTTCTGTCTGCTCGTTAGCTTTAATCAATCATGCC | |
| | CTGCCTTGTCCCTCTCACTCCCCAGCCCCACCCCTAAGTGCCCAAAGTGG | |
| | GGAGGGACAAGGGATTCTGGGAAGCTTGAGCCTCCCCCAAAGCAATGTG | |
| | AGTCCCAGAGCCCGCTTTTGTTCTTCCCCACAATTCCATTACTAAGAAACA | |
| | CATCAAATAAACTGACTTTTTCCCCCCAATAAAAGCTCTTCTTTTTTAATA | |
| | TATAAAAGCCCC | |
| MDK | GCAGGGTGCCCTGCAACTGGAAGAAGGAGTTTGGAGGTGAGGCGGGGCG | 7 |
| MDK | GCAGGGTGCCCTGCAACTGGAAGAAGGAGTTTGGA | 8 |
| MDK | GCAGGGTGCCCTGCAACTGGAAGAAGGAGT | 9 |
| MDK | CAACTGGAAGAAGGAGTTTGGA | 10 |
| MDK | CAACTGGAAGAAGGAGTTTGGAGGTGAGGCGGG | 11 |
| MDK | ACTGGAAGAAGGAGTTTGGAGGTGAGGCGGGGCGCAGTCAG | 12 |
| MDK | ACTGGAAGAAGGAGTTTGGAGGTGAGGCGGG | 13 |
| MDK | GAGTTTGGAGGTGAGGCGGGGCGCAGTCAG | 14 |
| MDK | GGCACCCTGAAGAAGGCGCGCTACAATGCTCAGTGCCAGGAGACCATCC | 15 |
| Region 9 | GCGTCACCAAGCCCTGCACCCCCAAGACCAAAGCAAAGGCCAAAGGTCA | |
| MDK | GGCACCCTGAAGAAGGCGCGCTACAATGCTCAGTGCCAGGAGACCATCC | 16 |
| Region | GCGTCACCAAGCCCTGCA | |
| MDK | GGCACCCTGAAGAAGGCGCGCTACAATGCTCAGTG | 17 |
| MDK | GTGCCAGGAGACCATCCGCGTCACCAAGCCCTGCA | 18 |
| MDK | AAGCAAAGGCCAAAGGTCAGCGAAAGGAGAAGGGG | 19 |
| MDK | CGCGCGTTGTAGATAAGGTGAAGAA | 20 |
| MDK | GTAGATAAGGTGAAGAAGGGCGGCC | 21 |
| MDK | TTTCCGCGAGGGCACCTGCGGGGCC | 22 |
| MDK | CTGCAACTGGAAGAAGGAGTTTGGA | 23 |
| MDK | CAACTGGAAGAAGGAGTTTG | 24 |
| MDK | AGAAGGAGTTTGGAGGTGAGGCGGG | 25 |
| MDK | CTCTCTCGCCAGCCGACTGCAAGTA | 26 |
| MDK | CCAGCCGACTGCAAGTACAAGTTTG | 27 |
| MDK | CCTGAAGAAGGCGCGCTACAATGCT | 28 |
| MDK | AGGAGACCATCCGCGTCACCAAGCC | 29 |
| MDK | AAGGCCAAAGGTCAGCGAAAGGAGA | 30 |
| MDK | GCAGGGTGCCCTGCAACTGGAAGAA | 31 |
| MDK | GTGCCCTGCAACTGGAAGAAGGAGT | 32 |
| MDK | ACTGGAAGAAGGAGTTTGGAGGTGA | 33 |
| MDK | GAGTTTGGAGGTGAGGCGGGGCGCA | 34 |
| MDK | GAGTTTGGAGGTGAGGCGGGGCGCA | 35 |
| MDK | GGCACCCTGAAGAAGGCGCGCTACA | 36 |
| MDK | AGAAGGCGCGCTACAATGCTCAGTG | 37 |
| MDK | GTGCCAGGAGACCATCCGCGTCACC | 38 |
| MDK | ACCATCCGCGTCACCAAGCCCTGCA | 39 |
| MDK | AAGCAAAGGCCAAAGGTCAGCGAAA | 40 |
| MDK | CAAAGGTCAGCGAAAGGAGAAGGGG | 41 |
| MDK | AGTGCCAGGAGACCATCCGCGTCAC | 42 |

TABLE 3
SSOs

| | | SEQ ID |
|---|---|---|
| Region ID | Sequence (5′-3′) | NO: |
| MDK H3A(−12+13) | UUCUUCACCUUAUCUACAACGCGCG | 43 |
| MDK H3A(-04+21) | GGCCGCCCUUCUUCACCUUAUCUAC | 44 |
| MDK H3A(+89+113) | GGCCCCGCAGGUGCCCUCGCGGAAA | 45 |
| MDK H3A(+143+167) | UCCAAACUCCUUCUUCCAGUUGCAG | 46 |
| MDK H3A(+146+165) | AAACUCCUUCUUCCAGUUGC | 47 |
| MDK H3D(+15−10) | CCCGCCUCACCUCCAAACUCCUUCU | 48 |
| MDK H4A(−12+13) | UACUUGCAGUCGGCUGGCGAGAGAG | 49 |
| MDK H4A(−04+21) | CAAACUUGUACUUGCAGUCGGCUGG | 50 |
| MDK H4A(+74+98) | AGCAUUGUAGCGCGCCUUCUUCAGG | 51 |
| MDK H4A(+106+130) | GGCUUGGUGACGCGGAUGGUCUCCU | 52 |
| MDK H4D(+10−15) | UCUCCUUUCGCUGACCUUUGGCCUU | 53 |
| MDK H3A(+133+157) | UUCUUCCAGUUGCAGGGCACCCUGC | 54 |
| MDK H3A(+138+162) | ACUCCUUCUUCCAGUUGCAGGGCAC | 55 |
| MDK H3D(+21−04) | UCACCUCCAAACUCCUUCUUCCAGU | 56 |
| MDK H3D(+10-−15) | UGCGCCCCGCCUCACCUCCAAACUC | 57 |
| MDK H3D(+05−20) | CUGACUGCGCCCCGCCUCACCUCCA | 58 |
| MDK H4A(+69+93) | UGUAGCGCGCCUUCUUCAGGGUGCC | 59 |
| MDK H4A(+79+103) | CACUGAGCAUUGUAGCGCGCCUUCU | 60 |
| MDK H4A(+101+125) | GGUGACGCGGAUGGUCUCCUGGCAC | 61 |
| MDK H4A(+111+135) | UGCAGGGCUUGGUGACGCGGAUGGU | 62 |
| MDK H4D(+15−10) | UUUCGCUGACCUUUGGCCUUUGCUU | 63 |
| MDK H4D(+05−20) | CCCCUUCUCCUUUCGCUGACCUUUG | 64 |

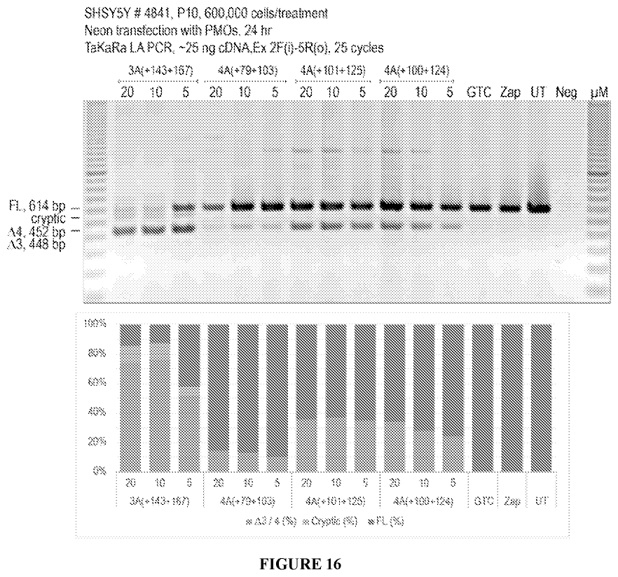

proliferation and dilution of the PMOs effects. The PMO SSO directed to exon 3 reached around 80% skipping efficiency, while the PMO SSOs directed to exon 4 reach around 40% skipping efficiency (see FIG. 16).

TABLE 6
List of PMO SSOs targeting exons 3 and 4 of MDK

| Oligo Label | AO Sequence (5′-3′) |
|---|---|
| MDK H3A(+143+167) | TCCAAACTCCTTCTTCCAGTTGCAG |
| MDK H4A(+79+103) | CACTGAGCATTGTAGCGCGCCTCT |
| MDK H4A(+101+125) | GGTGACGCGGATGGTCTCCTGGCAC |
| MDK H4A(+100+124) | GTGACGCGGATGGTCTCCTGGCACT |

### Example 7: Western Blot Evaluation

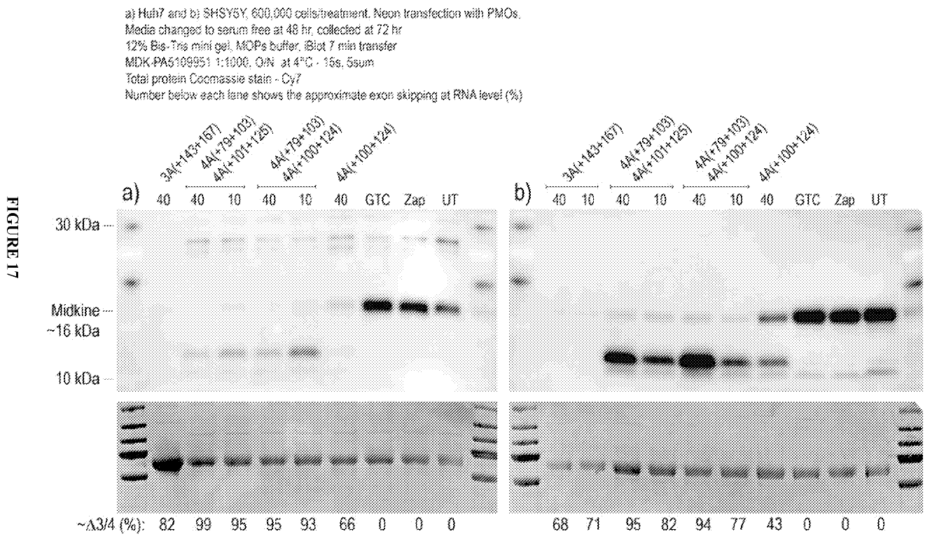

Huh7 and SHSY5Y cells were transfected with the most promising exon 3 and 4 AOs as both individuals and two-AO cocktails using lipofectamine 3000. The cells were incubated for 48 hours after transfection and then collected for RNA or protein analysis. RNA analysis revealed that the transfection was efficient with induction of both exon 3 and exon 4 skipping (FIG. 17, top panel). Western blotting clearly shows the reduction in full length midkine in the control lanes (GTC, ZAP, UTC) relative to the short form in all tracks with Exon 4 SSOs (FIG. 17, bottom panel).

FIG. 17 provides evidence of the production of a truncated human midkine protein with an exon 4 deletion.

It will be appreciated by persons skilled in the art that numerous variations and/or modifications may be made to the above-described examples, without departing from the broad general scope of the present disclosure. The present examples are, therefore, to be considered in all respects as illustrative and not restrictive.

1. A splice-switching oligonucleotide (SSO) which is between 10 and 50 nucleotides in length, the SSO comprising a nucleotide sequence of at least 10 contiguous nucleotides which is substantially complementary to a target region of corresponding length within a pre-mRNA sequence of human midkine.

2. The SSO of claim 1, wherein the SSO specifically hybridises to the target region of corresponding length within the pre-mRNA sequence of human midkine to disrupt splicing and thereby induce exon skipping of human midkine.

3. The SSO of claim 1, wherein the SSO specifically hybridises to the target region of corresponding length within the pre-mRNA sequence of human midkine to disrupt splicing and thereby induce exon skipping of exon 3 and/or exon 4 of human midkine.

4. The SSO of claim 1, wherein the target region is set forth in:
i) any one of SEQ ID NOs: 7-14, 20-25 and 31-35;
ii) any one of SEQ ID NOs: 20-25 and 31-35; or
iii) SEQ ID NO: 23.

5. (canceled)

6. (canceled)

7. The SSO of claim 1, wherein the SSO comprises a nucleotide sequence set forth in:
i) any one of SEQ ID NOs: 43-48 and 54-58; or
ii) SEQ ID NO: 46.

8. (canceled)

9. The SSO of claim 1, wherein the target region is set forth in:
i) any one of SEQ ID NOs: 15-19, 26-30 and 36-42;
ii) any one of SEQ ID NOs: 26-30 and 36-42; or
iii) any one of SEQ ID NOs: 37, 38 and 42.

10. (canceled)

11. (canceled)

12. The SSO of claim 1, wherein the SSO comprises a nucleotide sequence set forth in:
i) any one of SEQ ID NOs: 49-53 and 59-65; or
ii) any one of SEQ ID NOs: 60, 61 and 65.

13. (canceled)

14. The SSO of claim 1, wherein one or more nucleotides within the SSO are modified.

15. The SSO of claim 14, wherein all of the nucleotides within the SSO are modified.

16. The SSO of claim 14, wherein the SSO is a 2′-0-methyl phosphorothioate (2′-OMe-PS) SSO, a 2′-0-methoxyethyl (2′MOE) SSO, a locked nucleic acid (LNA) SSO, a morpholino oligonucleotide SSO, or a phosphorodiamidate morpholino (PMO) SSO.

17. The SSO of claim 1, wherein the SSO comprises a sequence of between 20-25 nucleotides which is substantially complementary to the target region.

18. A pharmaceutical composition comprising the SSO of claim 1, and a pharmaceutically acceptable carrier or diluent.

19. The pharmaceutical composition of claim 18, comprising a plurality of the SSOs according to claim 1.

20. The pharmaceutical composition of claim 19, wherein the plurality of SSOs comprises two or more of the SSOs selected from the group consisting of SEQ ID NOs: 43-65.

21. A method for inhibiting an interaction between human midkine and a ligand thereof on the surface of or in a cell, said method comprising exposing the cell to the SSO according to claim 1, or the pharmaceutical composition according to claim 18.

22. A method for inhibiting human midkine activity in a cell, said method comprising exposing the cell to the SSO according to claim 1, or the pharmaceutical composition according to claim 18.

23. A method for treating or preventing a midkine-related disease or disorder in a subject in need thereof, said method comprising administering to the subject the SSO according to claim 1, or the pharmaceutical composition according to claim 18.

24. The method of claim 23, wherein the midkine-related disease or disorder is an autoimmune disease, cancer, an inflammatory disease or multiple sclerosis.

25. The method of claim 24, wherein the midkine-related disease or disorder is cancer.

26. (canceled)

27. (canceled)

28. (canceled)

29. (canceled)

30. (canceled)
